(19) United States
(12) Patent Application Publication
(10) Pub. No.: US 2023/0344454 A1
(43) Pub. Date: Oct. 26, 2023

(54) ITEM STATUS TRACKING SYSTEM AND METHOD

(71) Applicant: NanoThings, Inc., New York, NY (US)

(72) Inventors: Timothy Williams, New York, NY (US); Gary Lomp, Centerport, NY (US); Friedrich Eibensteiner, Linz (AT); Josep Miquel Jornet, Clarence Center, NY (US)

(73) Assignee: NanoThings, Inc., New York, NY (US)

(21) Appl. No.: 17/982,936

(22) Filed: Nov. 8, 2022

Related U.S. Application Data
(63) Continuation of application No. 16/752,795, filed on Jan. 27, 2020, now abandoned.
Application No. 16/752,795 is a continuation of application No. 16/040,335, filed on Jul. 19, 2018, now abandoned.
Application No. 16/040,335 is a continuation of application No. PCT/US2018/017656, filed on Feb. 9, 2018.
Application No. PCT/US2018/017656 is a continuation-in-part of application No. 15/783,623, filed on Oct. 13, 2017, now abandoned.
Application No. 15/783,623 is a continuation-in-part of application No. 15/703,311, filed on Sep. 13, 2017, now abandoned.
(60) Provisional application No. 62/408,551, filed on Oct. 14, 2016.

(51) Int. Cl. H04B 1/04; H01Q 1/38; H01M 10/38; C09J 9/02; C09J 163/00; G16H 40/67; G06Q 10/087; G16H 40/20; H01Q 9/28

(52) U.S. Cl. CPC H04B 1/04; H01Q 1/38; H01M 10/38; C09J 9/02; C09J 163/00; G16H 40/67; G06Q 10/087; G16H 40/20; H01Q 9/285; A61B 2050/005

(57) ABSTRACT

A tracking device includes an antenna, a printed energy storage device, a transmitter powered by the printed energy storage device, and control circuitry configured to control the transmitter to transmit, using the antenna, information indicating a status detected by a sensor.

### CROSS-REFERENCE TO RELATED APPLICATIONS

The present application is a continuation of U.S. application Ser. No. 16/752,795, titled “ITEM STATUS TRACKING SYSTEM AND METHOD” filed Jan. 27, 2020, which is a continuation of U.S. patent application Ser. No. 16/040,335 titled “ITEM STATUS TRACKING SYSTEM AND METHOD” filed Jul. 19, 2018, which is a continuation of International Application PCT/US2018/017656, titled “ITEM STATUS TRACKING SYSTEM AND METHOD, filed Feb. 9, 2018, and a continuation-in-part of U.S. application Ser. No. 15/783,623, titled “A SYSTEM AND METHOD FOR TRACKING THE STATUS OF PARCELS AND MAILED MARKETING MEDIA AND REPORTING THE DISPOSITION THEREOF” filed Oct. 13, 2017, which is a continuation-in-part of U.S. application Ser. No. 15/703,311 titled “PRINTED TRACKING DEVICE, AND SYSTEM AND METHOD FOR USE IN A LOW POWER WIDE AREA NETWORK” filed Sep. 13, 2017, each of which is hereby incorporated by reference in its entirety. U.S. application Ser. Nos. 15/783,623 and 15/703,311 each claim the benefit under 35 U.S.C. § 119(e) to U.S. Provisional Application Ser. No. 62/408,551 titled “A SYSTEM AND METHOD FOR TRACKING THE STATUS OF PARCELS AND MAILED MARKETING MEDIA AND REPORTING THE DISPOSITION THEREOF” filed on Oct. 14, 2016, which is also herein incorporated by reference in its entirety. The present application claims the benefit of each of the above-identified applications.

### FIELD

Disclosed embodiments relate to a tracking and feedback system.

### BACKGROUND

Tracking systems may be used to track items as they move from one location to another.

### SUMMARY

A tracking device comprises an antenna, a printed energy storage device, a transmitter powered by the printed energy storage device, and control circuitry configured to control the transmitter to transmit, using the antenna, information indicating a status detected by a sensor.

A tracking device comprises a printed antenna, an energy storage device, a transmitter powered by the energy storage device, and control circuitry configured to control the transmitter to transmit, using the printed antenna, information indicating a status detected by a sensor.

The antenna may extend across a surface of a substrate.

The antenna may be planar.

The antenna may comprise a conductor deposited on a substrate.

The antenna may comprise first and second curved conductors.

The antenna may comprise first and second extensions of the first and second curved conductors, respectively.

The first and second extensions may extend toward one another.

The antenna may have an impedance of 50 ohms.

The antenna may be omnidirectional.

The antenna may be a dipole antenna.

The energy storage device may comprise a printed battery.

The printed battery may a carbon-zinc battery or a zinc magnesium dioxide battery.

The printed battery may be printed on the same substrate as the antenna.

The energy storage device may be disposable.

The energy storage device may be non-toxic.

The transmitter may be configured to transmit the information through a low-power wide area network (LPWAN).

The transmitter may be configured to transmit the information through an ISM band.

The transmitter may be configured to transmit the information in response to detection of the status by the sensor.

The transmitter may be configured to transmit the information in response to expiration of a timer.

The transmitter may be configured to transmit the information repeatedly for redundancy.

The transmitter may be configured to transmit health messages regarding the tracking device.

The transmitter may be configured to transmit the information and error correction information.

The transmitter may be configured to transmit the information to a receiver positioned more than 0.5 miles and less than one hundred miles from the transmitter.

The tracking device may be configured to store an identifier of the tracking device, destination address or recipient.

The tracking device may be configured to store a destination address for the information.

The destination address may comprise an internet protocol (IP) address.

The transmitter may be configured to transmit the identifier and/or the destination address along with the information.

The transmitter may be configured to transmit a packet comprising the information and the identifier and/or the destination address.

The information, the destination address and/or the identifier may be included in a payload of the packet.

The transmitter may be configured to send transmissions of less than 75 ms.

The tracking device may be configured to send the information only once in the lifetime of the tracking device.

The tracking device may be configured to determine its position and transmit its position.

The tracking device may be configured to determine its position using a global navigation satellite system receiver, or short-range wireless network positioning.

The tracking device may be configured to perform authentication by reading biometric information.

The tracking device may be configured to perform the authentication using a fingerprint reader.

Power may be disconnected to a portion of the tracking device until the sensor detects a change in the status, and the tracking device may be configured to provide power to the portion of the tracking device based on the detected change in the status.

The change in the status may comprise opening or closing a conductive path of the sensor.

The change in the status may comprise indicates opening of an item or removal of contents from the item.

The tracking device may further comprise a semiconductor die comprising at least a portion of the control circuitry.

The semiconductor die may be a bare semiconductor die without its own package.

The tracking device may be programmable through a wired or wireless interface.

The control circuitry may implement a finite state recognizer.

The control circuitry may be implemented by a programmed processor or dedicated logic circuitry.

The tracking device may be mechanically flexible.

The tracking device may be disposed on a paper substrate.

The sensor may be configured to detect opening or closing of a conductive connection.

The sensor may be configured to detect opening of an item, removal of contents from an item, or both.

The sensor may be configured to detect human interaction with a printed item.

The sensor may be configured to detect an environmental condition.

The sensor may be configured to detect opening of an item.

The sensor may be configured to detect removal of contents from an item.

The item may comprise a mailing, parcel, package or bound article.

The sensor may comprises a conductive path and the opening or removal of contents from the item may be sensed by opening or closing the conductive path.

The sensor may comprise conductors separated by an insulator.

The insulator may be configured to be removed when a force is applied to a leader.

When the insulator is removed, the conductors may come into contact with one another and close the conductive path.

The conductors may be maintained under a force that presses them together.

The tracking device may be configured to provide a current to the conductive path.

Power may be enabled to a portion of the control circuitry in response to detecting that an item has been opened or contents have been removed from the item.

A method of forming electrical connections through a plurality of pages of a bound article includes forming perforations in the plurality of pages at an edge of the plurality of pages, disposing a conductive material in the perforations, and binding the plurality of pages at the edge.

The conductive material may comprise a conductive adhesive.

The conductive adhesive may comprise a conductive epoxy.

The bound article may comprise a magazine.

A bound article comprises a plurality of pages bound at a binding, a conductive material at the binding, and a tracking device electrically connected to the conductive material.

The plurality of pages may be bound at an edge of the plurality of pages, the plurality of pages comprising perforations at the edge, wherein the conductive material is disposed within the perforations.

The binding may comprise a conductive adhesive that conducts current anisotropically.

The conductive material may make electrical connections through at least a portion of the plurality of pages.

The bound article may further comprise a first conductor disposed on a first page of the plurality of pages and a second conductor disposed on a second page of the plurality of pages, the first and second conductors being in contact with the conductive material.

The conductive material may comprise a conductive adhesive.

A bound article comprises a plurality of bound pages and a tracking device. The tracking device comprises an antenna, an energy storage device, a transmitter powered by the energy storage device, a sensor and control circuitry configured to control the transmitter to transmit, using the antenna, information indicating a status detected by the sensor.

A printed article, comprises a tracking device, the tracking device comprising: an antenna, an energy storage device, a transmitter powered by the energy storage device, and a sensor; and control circuitry configured to control the transmitter to transmit, using the antenna, information indicating a status detected by the sensor.

A package or printed article, comprises a sensor comprising a conductive material forming a conductive path. The sensor senses interaction with the printed article or package based on opening or closing of the conductive path.

The sensor may sense opening of the article or package.

The printed article may comprise a first page and a second page, the first page comprising a first conductor and the second page comprising a second conductor in contact with the first conductor when the printed article is closed, wherein opening the printed article disconnects the first conductor and the second conductor from one another.

The printed article or package may further comprise a member removably attached to the printed article or package, wherein removal of the member opens the conductive path.

The member may comprise a sticker or a scratch off material.

The sensor may comprises a first conductor and a second conductor that are electrically isolated from one another, wherein the first conductor and the second conductor are positioned adjacent to one another such that when a human finger contacts both the first conductor and the second conductor, an electrical connection between the first conductor and the second conductor is closed.

The printed article or package may comprise a recall notice.

The package may be sealed.

The package may comprise a medical device.

An item, comprises a band wrapped around the item, a sensor disposed on the band, the sensor comprising a conductor extending around the item, and a tracking device connected to the sensor.

The sensor may detect when the band is broken by sensing opening of a conductive path formed by the conductor.

The band may comprise paper.

The tracking device may be disposed on the band.

The item may comprise a magazine or a package.

A tracking method comprises detecting a status of a sensor using a tracking device comprising a printed energy storage device and/or a printed antenna, and transmitting the status over a wireless network. A tracking method comprises receiving, over a wireless network, a status of a sensor detected using a tracking device comprising a printed energy storage device and/or a printed antenna.

A method of forming a tracking device comprises printing an energy storage device on a substrate, printing an antenna on the substrate, and printing a conductor on the substrate.

The method may further comprise disposing a processor on the substrate.

Printing the energy storage device may comprise printing a battery.

The antenna may be formed by printing a conductive ink on a substrate.

The tracking device may have connections formed by low temperature soldering.

The tracking device may be formed by disposing the substrate on a carrier substrate and performing soldering to form connections of the tracking device when the carrier is disposed on the carrier substrate, and subsequently the substrate and carrier substrate are separated.

The carrier substrate may not be adhered to the substrate during soldering.

The soldering may be performed by disposing the substrate on a hot plate.

The hot plate may have hot spots in locations on which connections are soldered on the tracking device

The foregoing summary is provided by way of illustration and is not intended to be limiting.

### BRIEF DESCRIPTION OF THE DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 is a diagram of a tracking system according to one embodiment;

FIG. 2 is a block diagram of a tracking device according to one embodiment;

FIGS. 3A and 3B show examples of a printed antenna;

FIGS. 3A-3M illustrate shapes, simulation results, and radiation patterns for the printed antenna.

FIG. 4 shows a block diagram of a transmission chain;

FIG. 5 shows a block diagram of a digital portion of the transmission chain;

FIG. 6 shows a block diagram of an analog portion of the transmission chain;

FIG. 7 shows a block diagram of an RF processing portion of the transmission chain;

FIG. 8 shows an example of a packet structure;

FIG. 9 shows examples of status information;

FIG. 10 illustrates a system for tracking mailings, parcels and other items;

FIG. 11 shows a flow chart of a method of tracking status of a parcel;

FIG. 12 shows an example of sensor circuitry;

FIGS. 13A, 13B and 13C show diagrams of a tracking device associated with a parcel;

FIG. 14 shows an example of sensor circuitry that uses a sensing resistor;

FIG. 15A shows an example of a process that can provide a conductive connection to one or more individual pages of a magazine or other bound article;

FIG. 15B shows a cross section of a bound article having conductive connections extending between pages;

FIG. 16 shows an example of a sensor that can detect opening of an item;

FIG. 17 shows an interactive sensor;

FIG. 18 shows an example of a contact sensor that includes two conductive regions separated by a distance smaller than the width of a finger;

FIG. 19 shows another way of providing electrical contact to one or more individual pages of a bound article, such as a magazine, for example;

FIG. 20 shows another method for providing the technology, in a magazine application for example, is to utilize “belly bands”;

FIG. 21 shows an example in which a sensor may be used provide confirmation that a parcel or other article has been received; and

FIG. 22 shows an example of using a tracking device to monitor the integrity or opening of a medical device package.

FIG. 23 is a block diagram of an illustrative computing device.

### DETAILED DESCRIPTION

The present application describes techniques and apparatus for tracking the status of items. A tracking device can be embedded within or affixed to an item, and may include a sensor that detects a status of the item itself or its environment. In some embodiments, the tracking device may be small in size and low-cost, enabling use in a large number of applications, several of which are described herein. In some embodiments, the tracking device may be disposable, as it may be formed of non-toxic materials that allow it to be discarded without special handling. The tracking device may transmit the status detected by the sensor an identifier of the tracking device and/or other information via a wireless communication network. In some embodiments, the wireless communication network is a low-power communications network. The low-power communications network may enable transmissions over significant distances, which in some cases may span kilometers or miles, such as 0.5-100 miles. The transmitted data from tracking devices is received at any of a variety of communication nodes of the wireless network, allowing the received tracking data to be collected and used in a variety of ways. The system may be entirely automatic and requires no manual scanning by third party carriers.

A tracking device having one or more of the characteristics described herein can provide improvements over existing tracking technologies and enable new tracking applications not previously feasible. For example, a tracking device as described herein may be embedded within or affixed to an item such as a mailing or parcel and used to track its status, such as whether a letter or parcel is opened by the recipient. A tracking device can be used to track whether an item has been maintained in the proper environmental conditions, such as temperature, or humidity, or handled appropriately, such as being maintained in the correct orientation or avoiding being exposed to shock or vibration, for example. However, this is merely by way of example, as there are many possible types of status that can be tracked.

Also described herein are energy management techniques, including methods and practices that conserve battery life and minimize data transmission, and discloses techniques for the minimization of circuit size, complexity and power consumption.

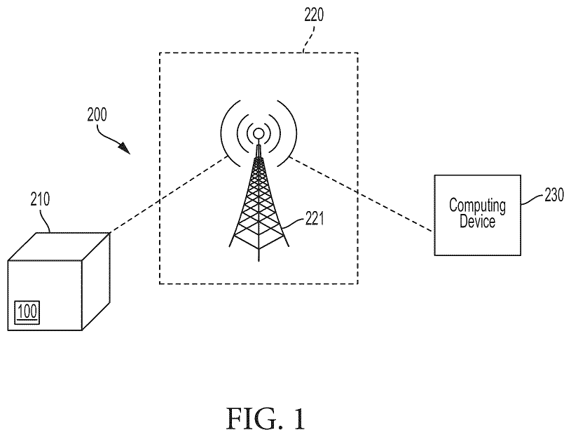

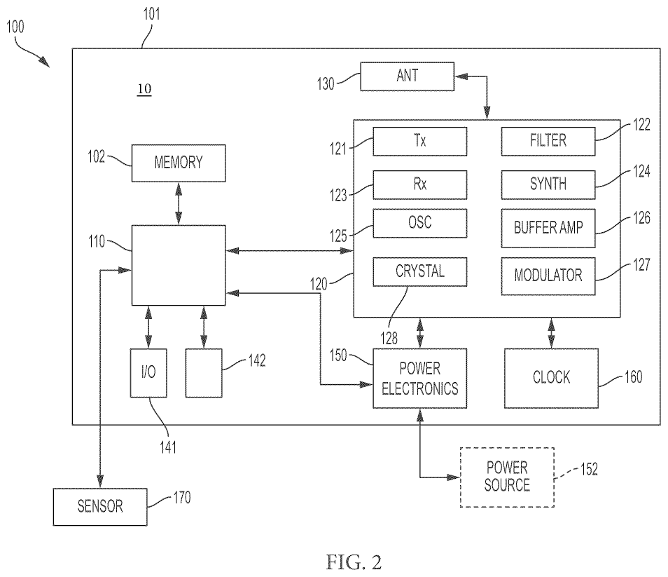

FIG. 1 shows an example of a tracking network 200 according to the present disclosure. In the tracking network 200 there is a tracking device 100 affixed to or embedded within an item 210, a wireless network 220, and a computing device 230, which may be a plurality of computing devices at the same location or in different locations. As discussed above, item 100 may be any type of item. By way of example and not limitation, the item 100 may be transportable object such as a mailing, a letter, a magazine, a container, a parcel, a box, an asset of any kind, a medical material or any other object. Items to be tracked may be shipped, mailed, or delivered through any suitable delivery network. Tracking device 100 is described further below in connection with FIG. 2. The tracking device may also store an identifier of the tracking device. The tracking device 100 may include or be connected to a sensor that detects the status of the item and/or its environment. For example, the tracking device 100 may detect whether a letter, parcel or other mailing has been opened, whether a person has interacted with some portion of a letter, parcel, mailing or magazine, such as an advertisement, and/or may detect any of a number of environmental factors to which the tracking device is subjected, examples of which are described herein. The tracking device 100 is configured to transmit information via network 200. For example, the tracking device 100 may be configured to transmit radio waves encoded with information such as the identifier of the tracking device and/or the status information detected by the sensor(s). In some embodiments, the network 200 may be a low-power wide area network (“LPWAN”). A LPWAN may enable low-power communications over significant distances. Some examples, without limitation, of suitable LPWAN are SIGFOX, long range radio “LoRA”, Weightless, Greenwave, LTE-MTC, Haystack, Ingenu Random Phase Multiple Access, and Narrow-Band Internet of Things (“N-B IoT”). Existing LPWANs use an open standard frequency Industrial, Scientific and Medical (ISM) band located at about 900 MHz in the United States and 868 MHz in Europe. These bands are intended to be exemplary and not limiting. ISM bands vary from nation to nation. The techniques described herein may be used for communication on any ISM band in any country, or any other suitable band. LPWAN can be advantageous for embodiments of the present disclosure because the tracking devices 100 can operate on low power while simultaneously having long range transmission capabilities. The tracking devices 100 disclosed herein are suitable for use in LPWAN at least in part because the tracking data transmissions may be relatively small. In some embodiments, tracking devices 100 may send less than 1000 bytes of data per day at 5000 bits per second or less. In some embodiments, the tracking device 100 may transmit less than 100 bytes or 1000 bytes during the lifetime of the tracking device 100. In some embodiments, the transmissions may be relatively short, such as less than 400 ms or less than 75 ms.

The system may take advantage of the emergent Internet of Things (IoT) paradigm. In some embodiments, by exploiting IoT platforms, especially certain wireless transport networks that are currently in development or deployed, the benefits of the present disclosure may be realized without the need for creating, developing and deploying extensive infrastructure. In some applications, such as determining the disposition of a mailing or parcel, the transmission of data may occur only once in the lifetime of the tracking device. In the present disclosure the network capacity may be accessed by a much higher geographical density of distinct transmissions. However, in some embodiments each transmitting element may be used only once, or, alternatively, is used a limited number of times for short durations, or even an unlimited number of times but at a very low percentage of the time (as in the tracking of non-perishable assets). By contrast, cellular and related infrastructure may be comparatively expensive to deploy and operate and may not be efficient for relaying large volumes of small disjointed (unrelated to one another) data packets from widely distributed sources.

In other embodiments, the tracking device 100 be adapted to send information to a local computing device over a suitable interface, such as a short-range wireless network, examples of which include WiFi and Bluetooth. In some embodiments, the tracking device may be adapted to send information over the conventional telephone network, perhaps automatically.

The encoded radio waves may be received by a base station 221 or another receiving device of the wireless network 220. The information transmitted may be extracted and sent to computing device 230 through any suitable wired or wireless network. In some embodiments, the computing device 230 serves as a logistics management client. The computing device 230 may include or be connected to a database correlating tracking device identifiers with particular items and information relating to the tracking device, items, such as a destination address, addressee, addressee contact information, etc. The tracking network 200 is capable of receiving, demodulating and decoding a radio signal transmitted by the tracking device 100 and extracting the information carried by the signal, e.g., information regarding the status of the item. The decoded payload may be transferred via a digital network interface to a digital carrier network, such as the Internet or another digital network capable of routing digital information (such as data packets), to the computing device 230. In some embodiments, the operator of the computing device(s) 230 may use information sent from the tracking device 100 for myriad purposes. The computing device(s) 230 can include memory or other storage so that information regarding the status of tracking device 100 can be stored and used in the future.

The computing device(s) 230 can employ an application interface (API) that is compatible with the wireless network 220 in order to perform the information retrieval from the wireless network 220. This API may be a conduit for data access stored in internal servers related to event codes and behaviors captured by the tracking network 200. In particular, if the tracking network 200 does not provide delivery of the feedback message to the computing device 230 via standard network routing techniques, e.g., an Internet using IP addresses, but rather stores the recipients' feedback datagram or datagram contents in a storage buffer, a method (utility or API) could be used to retrieve the contents from a storage location.

In some embodiments, the wireless network 220 may use the received signal to identify the location of the tracking device 100, and, thereby, the location of an item being tracked. This location information can be based on the known location of the receiving base station 221. Based on the known range of the wireless network 220, the location of the tracking device can be determined to be located near the base station at which the signal is received, within the range of the wireless network. In some embodiments, more sophisticated estimation of the location of the tracking device 100 may be implemented, such as by employing trilateration (or multi-lateration) methods. In other embodiments, the tracking device 100 may include a global navigation satellite system (GNSS) receiver, such as a global positioning system (GPS) receiver, to determine a location of the tracking device 100. However, in many embodiments in which low power consumption is desired, the tracking device 100 may not include a GNSS receiver. In some embodiments, the tracking device 100 may have a short-range wireless transceiver, such as a WiFi transceiver and/or a Bluetooth transceiver, and may determine its position using the sort-range wireless transceiver, e.g., by WiFi and/or Bluetooth positioning.

Some embodiments of the present disclosure include biometric authentication technology in the tracking device 100, such as fingerprint reading and identification technology, e.g., a fingerprint reader, which can provide added security and authentication capabilities. A biometric reader such as a fingerprint reader may be connected to the processor 110, allowing the biometric reader to enable or disable various functions by the processor 110. Again, however, in embodiments in which low power consumption is desired, the tracking device 100 may not include such capabilities.

FIG. 2 depicts a block diagram of a tracking device 100 that can be used in any or all of the use cases described herein as well as other use cases. The tracking device 100 includes a circuit 10 disposed on a substrate 101. The substrate 101 may be formed of any material, including rigid or flexible materials. In some embodiments, the substrate 101 may be a thin film, such as a PET film or a paper film, a cardboard member (e.g., a portion of a cardboard box), a plastic member, a paper member, or a composite wood member, by way of example. In some embodiments, the substrate 101 may be the item 210 or part of the item 210 or its packaging. For example, in some embodiments discussed further below, the substrate may be a printed article such as a mailing, a letter, a magazine, etc. In some embodiments, a portion or all of the circuit 10 may be printed on the substrate 101. Features of the circuit 10 may be defined by additive or subtractive methods such as printing or etching, respectively. However, any suitable technique may be used to form the circuit 10, including known techniques for forming conductive structures. In some embodiments, the substrate 101 may include a printed circuit board and a portion of the circuit 10 may be formed on the printed circuit board. Wiring between circuit components may be in the form of traces on the printed circuit board or other substrate. In some embodiments, the substrate 101 is designed to be flexible and thin so that it can be securely attached to items such as magazines, marketing mailings or other flexible items.

The circuit 10 may be coupled to a sensor 170. As shown in FIG. 2, the sensor 170 may be disposed off the substrate 101. However, in some embodiments, the sensor 170 may be disposed on the substrate 101, as the tracking device 100 is not limited as to the position of the sensor 170 relative to the substrate 101. Further, the sensor 170 may be separate from the circuit or may be part of the circuit 10, as the sensor 170 need not be considered as separate from the circuit 10. In some embodiments, the sensor 170 may be a printed sensor. Examples of printed sensors are described herein.

The circuit 10 may include a memory 102, a processor 110, a transmitter 120. A portion of circuit 10, such as one or more switches, may be realized by a semiconductor chip disposed on the substrate 10. In some embodiments, the processor 110 and/or memory 110 are disposed on such a semiconductor chip. The semiconductor chip may be incorporated into the tracking device 100 as a bare die without a package of its own, and packaging may be provided by layers of the tracking device 100 (e.g., one or more films). However, in some embodiments the semiconductor chip may be packaged (e.g., in a QFN or other packaged die form) and then incorporated into the tracking device 100.

In some embodiments, the transmitter 120 may be a transceiver capable of both transmission and reception of signals. In some embodiments, the circuit 10 may include a receiver (not shown) in addition to the transmitter 120. The circuit 10 may also include power electronics 150 and an antenna 130. In some embodiments, the memory 102, which could be a RAM, a flash memory, or any other memory, is communicatively coupled to the processor 110. The memory 102 may store an identifier of the tracking device 100. In some embodiments, the identifier may be specific to the tracking device 100, the item 210, an address, person or entity to which the item is addressed or otherwise associated. A destination address for packets sent by the tracking device via the wireless network 220 may also be stored in the memory 102. The identifier may be stored in the memory at any suitable time, such as during or after manufacturing. The information may be read and stored in the memory in any of a variety of ways, such as through an electrical interface, optical scanning, or another technique. The information may be transferred using a wired connection or a wireless connection, such as via WiFI, Bluetooth, RFID or NFC for example. The tracking device 100 may include a suitable wired or wireless interface to receive the programming information. Alternatively, the programming may be arranged in a manner that allows the transponder to receive digital information wirelessly by being placed in the field of a programming instrument. Information may be stored in memory 102 either encrypted or in plaintext.

The processor 110 may control the tracking device 100. The processor may perform various functions, including programming the tracking device 100 for a specific use case (such as the sensor configuration: e.g., temperature, pressure and/or packaging state); provisioning the tracking device 100 to correlate and configure its transmissions/reports with a database and a mission profile (e.g., pairing: association of the tracking device 100 with an address, as in mass mail applications, a user ID or an article ID); entering mission parameters such as report intervals and number, timer settings, etc. Other functionality may including controlling the operation of the digital subsystem that creates the encoded digital (binary) data payload and formats it for digital RF communication. This may include forward error correction algorithms or other similar methods for increasing the reliability of the RF transmission process. Other functionality may include monitoring the status of the tracking device 100 such as timers, battery, and/or sensors, and storing the data in local memory registers; decision processing such as comparing measurements with set thresholds, triggering reports, etc. The processor may include/execute basic signal generation algorithms such as spectrum control (filtering), signal level control, and/or turning the radio transmitter on/off. The processor 110 may be a programmed processor, such as a microprocessor or microcontroller that executes program instructions. However, processor 110 need not be a programmed processor, and in some embodiments may be implemented by application-specific circuitry.

In one embodiment, the programming of the tracking device 100 is performed using a serial interface in which a bit sequence is applied to the processing element through an input-output (I/O) terminal 141. Devices can be manufactured with the capability of supporting any of a plurality of applications. The multiplicity of capabilities is described by the types of sensors available in the application deployment, while the logic that controls the reporting is described by internal digital logic that is configurable through either manufacturing parameter settings (hard coding) or through the aforementioned serial I/O terminal 141. In certain embodiments the multiplicity of sensor types and configurations, especially the external sensor circuitry, is managed separately from the multiplicity of reporting and monitoring settings. The reporting and monitoring settings may be controlled through digital logic. When the digital processing element of the transponder is programmed, the bit pattern input on the serial port induces, or instantiates, a particular configuration of the control logic inside the chip (digital circuit). This configuration can be stored in memory, and the particular configuration selected via a memory address or offset or block of addresses.

In another embodiment, the programming of the digital control can be based on finite state recognizer technology. This type of programming is useful when it is desired to make the transponder especially small. In particular, the digital processor may be designed without a software processor, thereby reducing cost and complexity of the digital subsystem. Finite state recognizer technology is known in the art of computer engineering, especially in the art of compiler design. In short, this technique operates by accepting a string or stream of tokens (in some embodiments of the present disclosure the tokens are simple binary symbols represented by voltages or currents injected through an I/O pin or terminal or port) and sequentially processing said string or stream in order to “parse” the “string sentence” into meaningful directives. Such a stream processing technique can be realized by defining certain bit patterns that are recognized and which cause a state machine to transition into desired control states. In one embodiment, the state can be a pointer to a segment of microcode. In another embodiment the state can be a register word that controls selected primitive functions that are predefined at device (chip) manufacturing time but which are only instantiated as a result the programming. It will be clear to one of ordinary skill in the art of computer programming, especially microcontroller and similar device programming (firmware programming), that the above method can be designed in a manner that removes the need for a resident software control program and associated general purpose processing elements (CPU, memory, etc.). The benefit of this type of design is minimization of chip complexity (gate count, logic blocks, number of transistors, etc.) and power consumption.

In another embodiment, which is at the other end of the complexity spectrum, programming of the intelligent transponder is achieved by simply loading a program file into a transponder that is equipped with a CPU and memory and any peripheral (such as I/O) components need for general purpose computer operation. In such an embodiment no special logic design aimed at minimization of logic complexity is required. The system logic is resident in the externally created control program. Such embedded system programming is also well understood in the art of digital systems design and related art. Such programming would, for example, set the frequency of temperature reporting if a temperature sensitive item is being tracked. As another example, the temperature threshold for which such monitoring should generate an alarm and report would be programmable.

The processor 110 is communicatively coupled to the transmitter/transceiver 120 and power electronics 150. The transmitter 120, which is also communicatively coupled to the power electronics 150, can be further comprised of one or more of the following: a transmit block 121, a receive block 123, a filter 122, a synthesizer 124, an oscillator 125, a buffer/amplifier 126, a modulator 127, a crystal 128 and a power source 152.

In some embodiments, the tracking device includes a power source 52. The power source 152 may include any suitable energy storage device, such as a battery or a capacitor (e.g., a supercapacitor). The energy storage device may be charged prior to deployment to provide energy for operations of the tracking device 100. In some embodiments, the power source 152 may also include a power source that provides energy to the energy storage device, such as a photovoltaic device or a device that harvests energy (e.g., from movement, vibration, ambient light, electromagnetic radiation or otherwise). The power source 152 may be disposed off the substrate 101, as shown in FIG. 2, or may be disposed on the substrate 101, as the techniques and devices described herein are not limited as to the location of the power source 152.

Advantageously, in some embodiments, the power source may include a printed energy storage device, such a printed battery. A printed energy storage device may have the benefit of low cost and small form-factor. In some embodiments, the energy storage device may be disposable. For example, the power source 152 may include a battery, such as a printed battery, formed of disposable materials. Examples of disposable battery material systems include carbon-zinc, and zinc magnesium dioxide. A disposable power source 152 may allow the tracking device 100 to be discarded without special handling. By contrast, many conventional batteries such as lithium-ion batters may be required to be recycled or disposed of with special handling in many jurisdictions. A disposable energy storage device, by contrast, faces no such restriction and may be discarded along with item 100 or its packaging.

Printing of a printed energy storage device can be arranged so that the substrate 101 is compatible with the handling of a parcel or item containing the tracking device 100. Thus, printing on semi rigid substrates 101 such as circuit boards may be appropriate when rigid packaging and tracking device 100 construction is used. In other embodiments, the printing may be applied to fabrics that are flexible, such as Mylar, or other flexible substrates.

Advantageously, in some embodiments antenna 130 may be a printed antenna (e.g., printed on substrate 101). Antenna 130 may be a loop or dipole antenna formed from a length of conductive material or dielectric material. The shape, thickness, and composition of the antenna 130 will determine its radiation behavior, including the directivity and gain. In some embodiments, the antenna 130 may be non-directional so that transmission to a base station does not depend on the particular position or orientation of the parcel or its packaging, to which the antenna 130 is embedded or attached.

In some embodiments, it may be desirable to utilize physically small antenna 130 geometry in which antenna 130 features, including overall length or area, are smaller than the operating radio frequency wavelength. In such embodiments, the substrate 101 may be configured to provide a ground plane with respect to which the antenna 130 operates. In some embodiments, the ground plane may be an integral part of the mechanical design of substrate 101 as well as its electrical design. In some embodiments, the addition of a ground plane may be achieved by creating a “sandwich” structure in which the ground plane is inserted between two outer substrates 101. In such an embodiment, the ground plane may be shared by antennas 130 printed separately on the two sides of the sandwich. This has the advantage of allowing smaller antenna 130 features while simultaneously providing antenna diversity.

In some embodiments, the antenna 130 will provide minimal or zero gain. In these embodiments, the RF communication function could be designed to close the radio link with low or no gain for antenna 130. Low or no antenna gain may be the result of the constraints on the antenna size and shape and/or lack of a ground plane. For example, in some embodiments the antenna may be two-dimensional (e.g., flat), and the fabrication process may not include an layer for a ground plane. In fact, the RF link analysis includes and provides margin for significant obstruction of the radio signal in its propagation from the parcel to the wireless network receiving antenna. However, in some embodiments the antenna may have gain, which may improve the distance signals can be transmitted and/or reduce the power consumed by transmissions.

As in the case of the battery, the choice of antenna material and printing method can be determined in accordance with required sturdiness, rigidity, flexibility and other environmental factors. Both conventional circuit board materials and nonconventional materials or substrates and processes, including flexible dielectric and or conductive fabrics, etching of conductive fabrics, deposition of conductive films, glues or paints, and similar methods may be used.

The antenna 130 may be a single antenna or a plurality of antennas. A plurality of antennas may produce signals directed in more than one direction of signal propagation. If a plurality of antennas are used to transmit signals, a device for switching, dividing or combining energy from plural antennas may be included, such as a switch, commutator or multiplexor. Antennas in any of these embodiments could be single band, dual band, broadband, directional, omni-directional, high gain, low gain, and any combination thereof. In yet an additional embodiment, the circuit 10 and the antenna 130 are coplanar.

The antenna may be planar and may be designed to radiate efficiently with a pattern normal to the plane in which it lies. The antenna may be as compact as possible and able to be constructed in a fabrication process at the same time as the overall tracking device is printed. Thus the antenna may be located on a substrate, which is, for example, Polyethylene terephthalate (PET).

The antenna has the following aspects.
1) The antenna radiation efficiency (gain)
2) The antenna input impedance (matching to the amplifier)
3) The antenna trace conductivity (should be as large as possible)
4) The antenna size and shape (affects Tag size)
5) The method of coupling the antenna to the RF signal source (an amplifier output terminal)

A printed antenna may be formed by depositing conductive ink on a PET substrate.

In addition to the preceding, in order to produce radiation efficiently the antenna may exhibit resonance at the radio frequency of operation. In the case of a single conductor antenna, such as a patch, a ground plane may be used. Since there is not a convenient ground plane available in a PET substrate, or other preferred substrate, a dipole structure is may be used.

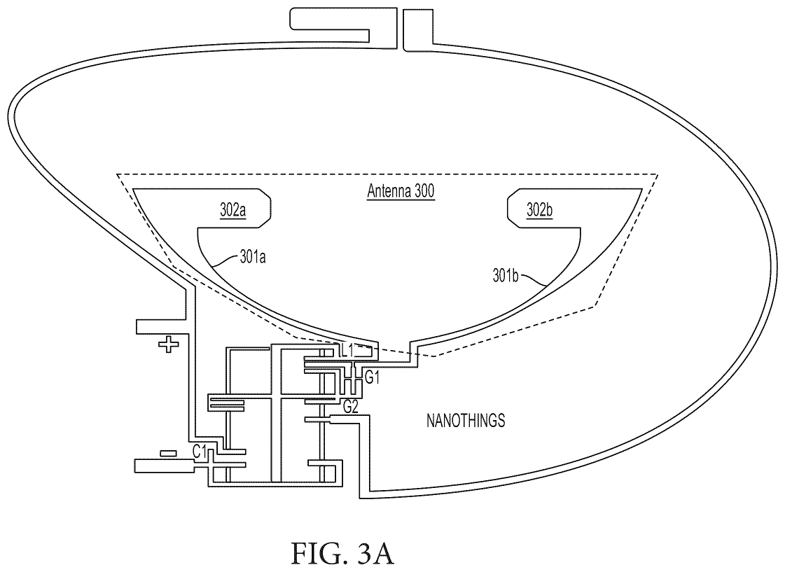

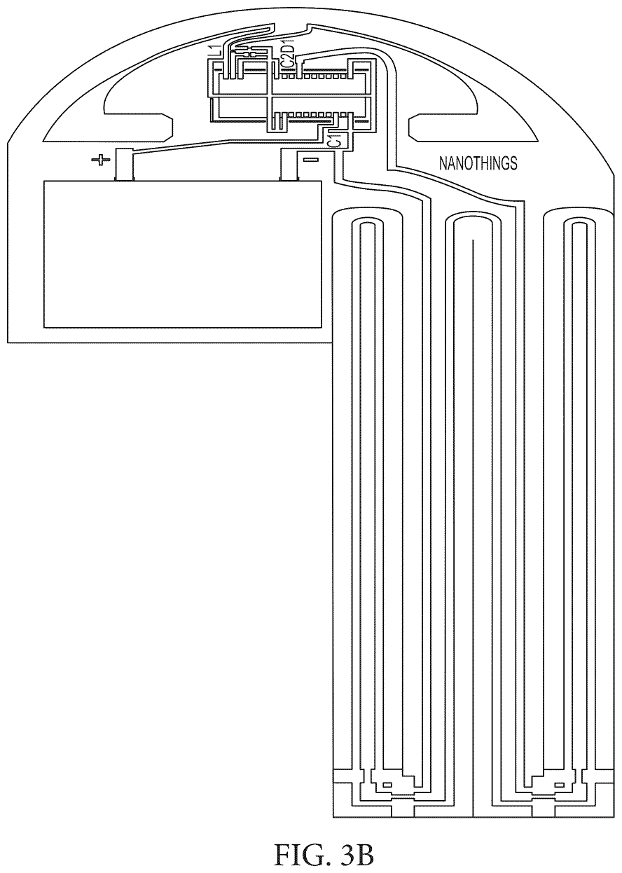

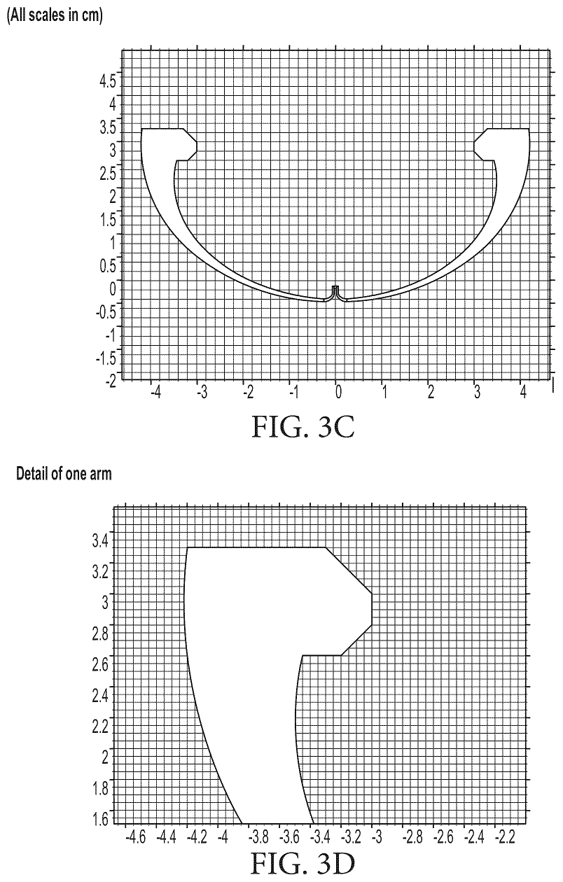

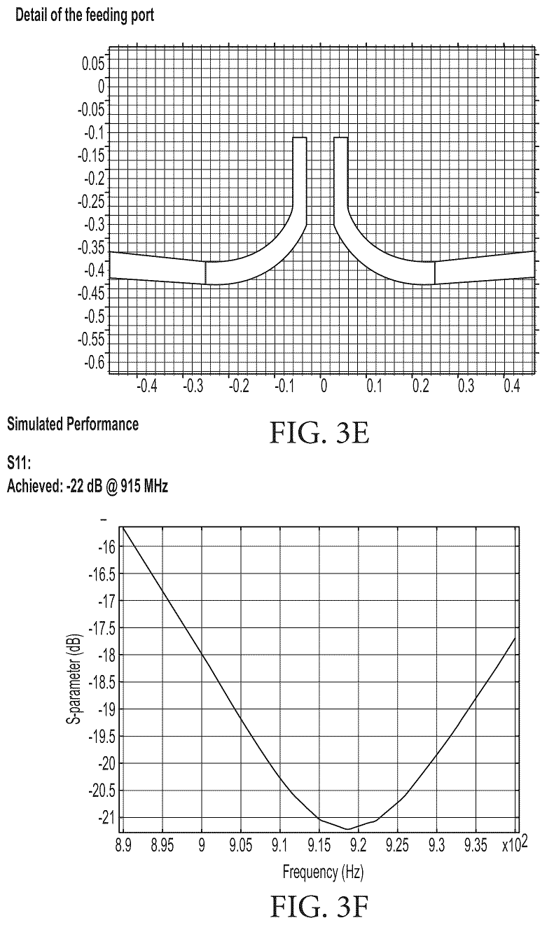

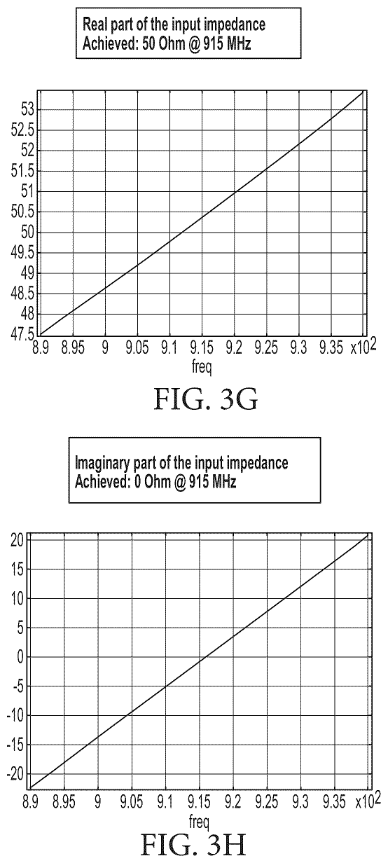

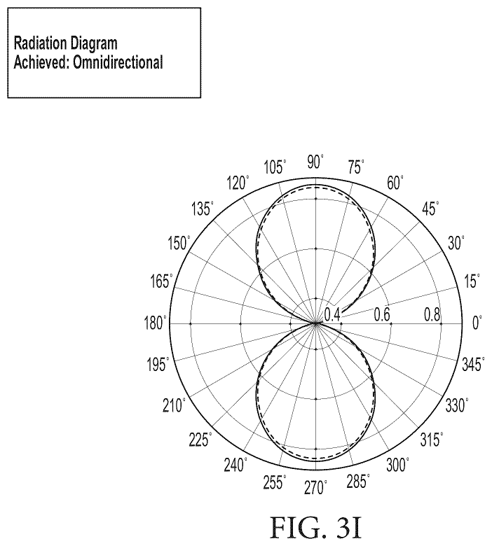

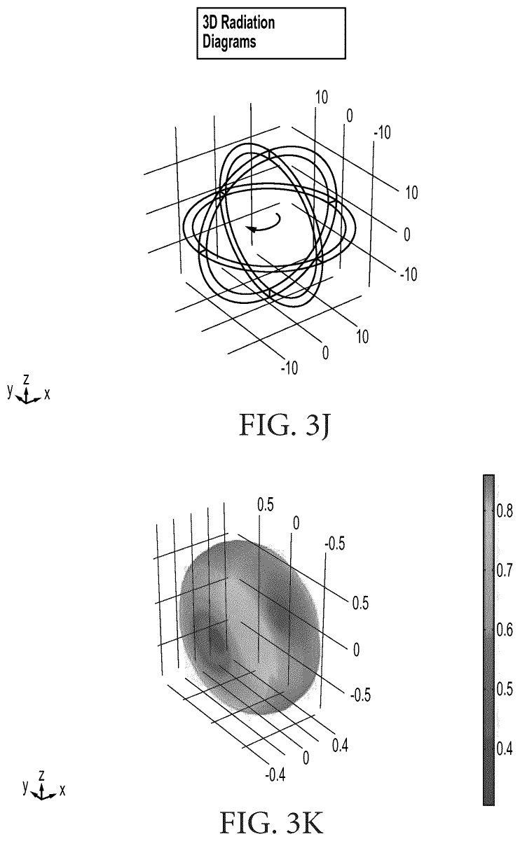

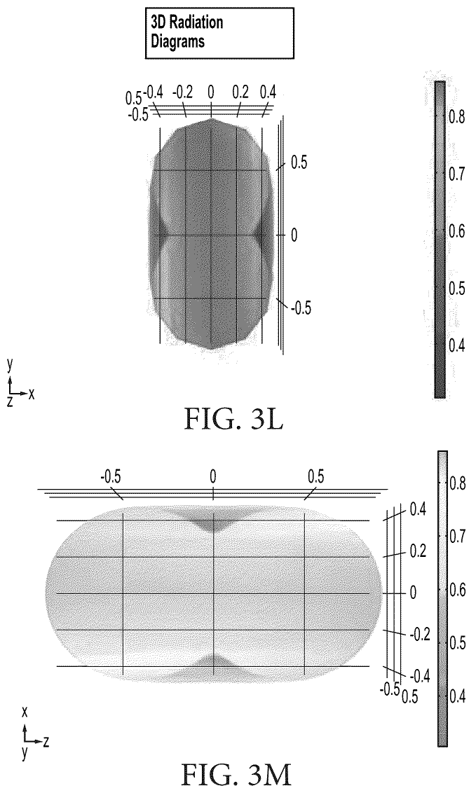

FIG. 3A shows a printed antenna, curvilinear, with a length equal to one half wavelength (two quarter wavelength sections), formed by depositing conductive ink on the substrate. In order to couple the antenna smoothly (without signal reflection at the junction) the antenna is tapered so that the end of the conductive segment is matched in physical size to the terminal (such as a pin on an integrated circuit, or other metal electrode) to which it is bonded (e.g., soldered). Since this results in a reduced width of the trace (lower conductivity) the taper is allowed to broaden along the segment of the dipole resulting in a greater mass of conductive material and low resistivity. Low resistivity produces low loss in the antenna and increases radiation efficiency. The curve of the antenna is determined so that the desired radiation behavior occurs. A conventional dipole can be used as a reference and the antenna is adjusted to be as close as possible to the reference. The curvature of the antenna may be limited. Careful analysis of the antenna electrical and radiation characteristics leads to the design shown in FIG. 3A.

The antenna shown may provide a purely resistive (no reactive component) 50 ohm input impedance. This is achieved by controlling the curvature, the taper of the trace thickness and the segment nodes (the ends of the segments). Further tailoring of the characteristics is achieved by providing the indicated notches at the rounded end nodes.

In order to allow the antenna to be printed in a high speed process the ink deposition should be able to cure quickly. In an embodiment, the antenna is designed for 14 micron thickness. Conductive inks may be used such as DuPont 5028 or DuPont 5029. However, other inks and thickness could be used. A thinner deposit of conductive material is possible if a material with a higher density of conductive particles is used so the resulting antenna resistivity is small even with the thinner trace.

The specific shape of the antenna may be altered. At least the shape could be mirrored. Other modifications are possible, including the following.
a. Gently Curved, no corners
b. Nodes at endpoints to decrease overall resistance
c. End nodes with notches to adjust impedance
d. Taper to optimize the junction to the RF signal source

FIGS. 3a and 3b show examples of printed antennas and associated circuitry. The horseshoe-shaped conductors in the center of FIG. 3A are the antenna 300. In this example, the antenna 300 has two curved conductors 301a, 301b that may be formed on a two-dimensional surface. The curved conductors terminate with extensions 302a, 302b, respectively, that are directed toward each other and the center of the antenna 300. In some embodiments, the antenna 300 may be a dipole antenna. However, the techniques described herein are not limited in this respect, as other types of antennas may be used. In some embodiments, the antenna 300 is shaped and sized to have an impedance of 50 ohms. However, in other embodiments, the antenna 300 may be shaped and sized to have a different impedance.

In some embodiments, the antenna 300 may have the following specifications.
Frequency band: 915 MHz (902-928 MHz)
Impedance: 50 Ohm
Ink Resistance: 12 mOhm/square at 25 microns thickness
Ink Thickness: 14 micrometers
Ink Conductivity: [1/(Resistance*Thickness)]=3.33e6 S/m

FIG. 3C shows the antenna 300 on a scale of cm. FIG. 3D shows further detail of an extension 302a, illustrating the extension is tapered toward a flat end at the right side (scale of cm). FIG. 3 shows the feeding port in further detail (scale of cm). FIGS. 3F-3H illustrate simulated performance of the antenna 300. FIGS. 3I-3M illustrate radiation diagrams of the antenna 300.

Returning to discussion of the FIG. 2, the circuit 10 may include a clock 160. The clock 160 may be used to measure a timeout event, or as an alarm or timer. A timer may be set at the time the tracking device is programmed. Clock 160 may provide relative or absolute time or time interval information. For example, the tracking device 100 may be configured to transmit status information at regular intervals. In some embodiments, the tracking device 100 may periodically send messages to indicate it is still within range of the network. Such messages may be termed “health messages.” Optionally, status information regarding the tracking device 100 may be included in the health messages. The clock 160 may be used to time the intervals between transmissions. As another example, the tracking device 100 may be configured to obtain measurements from the sensor 170 at regular intervals. Accordingly, the clock 160 may be used to time the intervals between measurements.

In some embodiments, there may be an RFID element, RF element, Wi-Fi element, Bluetooth element, cellular element, and the like coupled to or added onto the substrate 101 and/or circuit 10. In some embodiments, the circuit 10 may include input/output terminals 141, an A/D and/or a D/A converter 142.

The processor 110 is configured to receive input information from the sensors 170. Upon receipt of sensor 170 input, the processor 110 can generate an event code. The sensor 170 may be a single sensor or a plurality of sensors. The sensor(s) 170 may be selected to measure any of a variety of events, conditions or other status of the item 210 such as the opening of a package, breaking a seal, movement, temperature variation, acceleration, vibration, humidity, pressure, g-force, smoke detection, fire detection, gas detection, light, sound, orientation, stacking height, weight, or other status information. In embodiments in which the sensor 170 includes a plurality of sensors, each sensor may have an associated identifier.

In some embodiments, the above system elements are implemented in a low cost, low form factor and low power consumption manner. There are many techniques available for achieving these criteria. One is to utilize significant custom circuitry rather than “off the shelf” components that are designed for other general purpose applications such as microprocessors or self-contained radio transmitters. In some embodiments, the tracking device 100 can be a custom-made, single purpose device. In embodiments, at least a portion of the custom circuitry may be placed on a silicon chip or die, i.e., an ASIC. However, in some embodiments, the circuit 10 may include one or more “off the shelf” components, such as a microprocessor and/or memory.

In the art there are technologies for manufacturing custom ASICS. Furthermore, these technologies permit “mixed” analog, digital and radio frequency (RF) circuitry to be placed on a common substrate 101 or within a highly integrated miniaturized package. There are available, and may become available, other technologies different from silicon-based (or other semiconductor) chip design and manufacturing processes. These may include processes that print circuits on fabrics by painting or other deposition processes or processes that create circuitry by etching processes. The techniques described herein may use any such particular manufacturing and design alternatives and no particular manufacturing or design alternative is intended to be required for or limit the teachings of the present disclosure.

The present disclosure has many potential applications and these various applications may collectively entail significantly distinct physical “form factors” such as size, weight and power requirements. In view of this diverse application spectrum it will be appreciated that the tracking device may be embodied in a single highly integrated package to minimize size, weight and power requirements, as well as cost, or that the tracking device may be provided in “semi-knock-down” (SKD) form, in which case the components of the present disclosure are separated into major blocks, such as the RF electronics module, battery/power source, antenna, and sensors. These blocks may be adjusted to accommodate various applications consistent with the present disclosure. For example, the power source for some applications, like pallet or container tracking, may be made larger so that the usable active life of the transponder matches the pallet/container lifetime or in-service time interval. In other scenarios, such as direct mail, the power source may be made very small, consistent with the application only entailing a small number of active operating/transmission intervals.

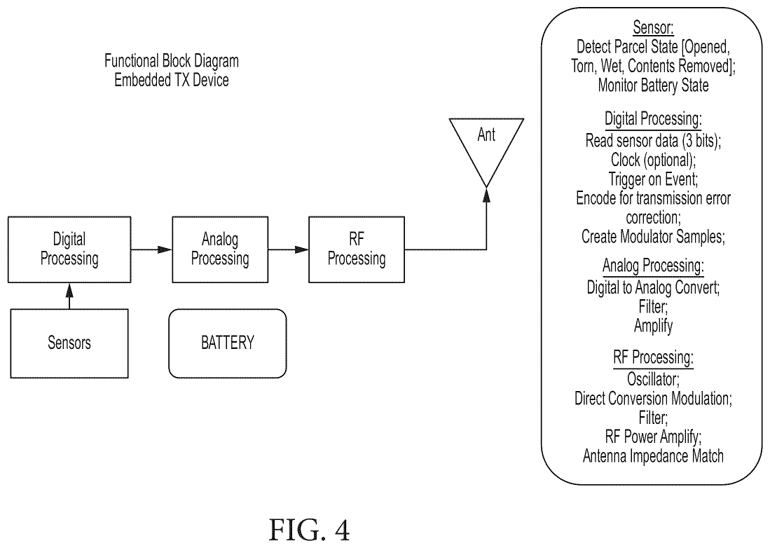

FIG. 4 shows an example of transmission signal chain that may be implemented by the tracking device 100. As illustrated in FIG. 4, information from the sensors may be provided to digital processing circuitry, which may include a processor 110 or dedicated digital circuitry. The digital processing circuitry may create a packet or a plurality of packets to be transmitted.

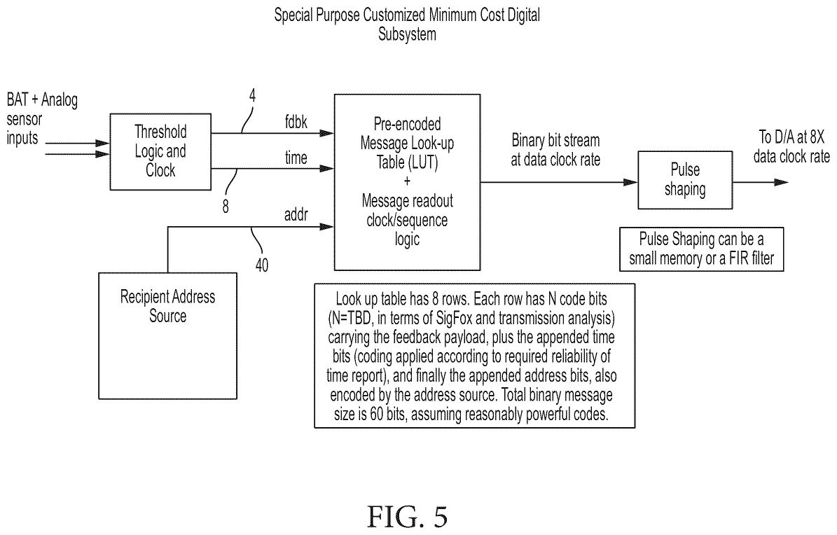

FIG. 5 shows an example of the digital processing circuitry. If the sensor(s) provide analog signals, they may be converted to digital signals by an A/D converter, or compared to one or more thresholds (e.g., using a comparator).

A clock may be used to set the timing of transmissions. At an appropriate time, the detected sensor input converted to digital format is used to look up an event code in the look up table. The digital processing circuitry may then produce a packet, which may include the event code and/or an identifier of the tracking device 100. Pulse shaping may be performed by a digital filter, such as an FIR filter, for example.

In some embodiments, if there is only one event of interest, such as “parcel opened”, then there is no need for an event decoder or encoder or register, or other “mapping” to map the detected event to an action to be relayed or reported by the transponder.

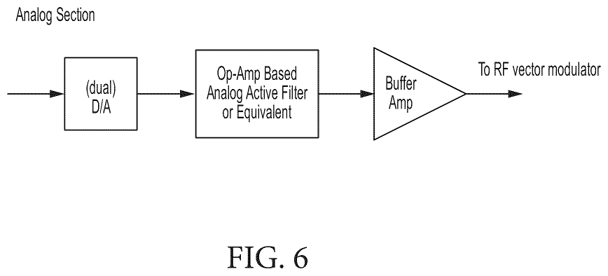

FIG. 6 shows an example of the analog processing circuitry. The packet from the digital circuitry may be converted into analog format by the D/A converter. Analog filtering may then be performed, e.g., by an op-amp based active filter. The resulting signal may then be buffered by a buffer amplifier.

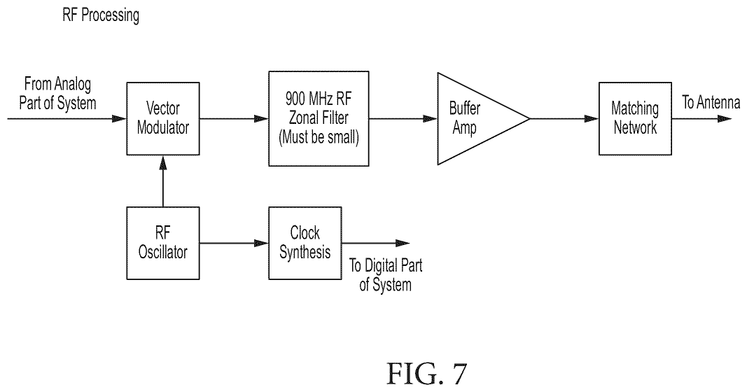

FIG. 7 shows an example of the RF processing circuitry. The analog signal from the analog processing circuitry may be modulated using any suitable modulation scheme. As shown, the signal may be modulated by a vector modulator. The modulation may be in response to an RF oscillator that oscillates at a carrier frequency. The resulting modulated signal may be within a suitable frequency band, such as the ISM band, for example. In the example illustrated in FIG. 7, the signal is modulated into the 900 MHZ band. The signal may then be bandpass filtered and buffered by a buffer amplifier. The signal output from the buffer amplifier is then provided to a matching network that matches the output impedance of the buffer amplifier to the antenna. The signal is then provided to the antenna for transmission.

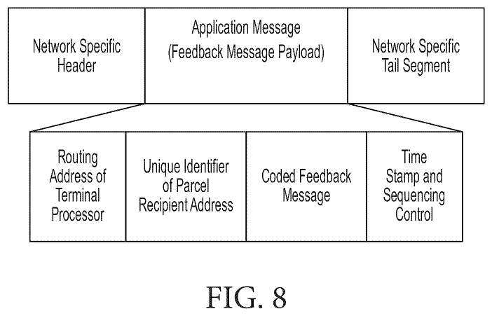

FIG. 8 shows an example of a packet transmitted by the tracking device 100, according to some embodiments. There may be more than one layer of information coding and or formatting applied to the message. One layer of coding and or formatting may be dictated by the wireless network operator and associated signal specifications. For example, packet header and tail information may be specified by the wireless network operator. Additional layers of coding are applied to enhance and or measure end-to-end message integrity, such as error correction coding, as discussed further below.

The information transmitted, according to the present disclosure, may be done using a signal of minimal complexity, making the signal generation and transmission technology (circuitry, firmware and software) simple and inexpensive. The packet may include a header or preamble portion, which may be suitable for a particular wireless network 220. The header or preamble portion may be used by the wireless network for synchronization and identification purposes. A non-information bearing stabilization preamble may be provided so that the radio frequency circuits have time to stabilize before the information bearing portion of the radio signal, i.e., header, preamble, and payload, is passed through the circuit.

The payload portion may include a destination address, which may be an address of the computing device 230 which receives the data. The payload portion may include an identifier of the item 210 and/or tracking device 100. The payload portion may include status information regarding the item 210 detected by the sensor 170. The status information may take the form of a digital code that indicates the status.

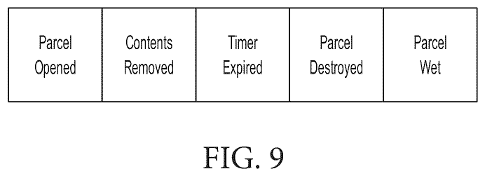

FIG. 9 shows examples of various types of status information, including an indication of whether a letter or parcel is opened, an indication of whether contents of the letter or parcel have been removed, an indication that a timer has expired, an indication that a parcel or mailing has been destroyed and/or or an indication of whether a parcel is wet. However, there are many other types of status information that may be provided, as discussed herein. In some embodiments, each status may be indicated by a single bit. However, the techniques described herein are not limited as to the number of bits used to indicate information. The status information, such as the information illustrated in FIG. 9, may be used to point to an appropriate memory location (hardware implementation) or software routine (software implementation), so that the appropriate action can be taken based on the current status.

Returning to the discussion of FIG. 8, the payload portion may include information regarding the timing of the status information. For example, the timing information may include a time stamp indicating when the sensor 170 detected a change in status, or a time at which the packet is transmitted. In some embodiments the timing information may include sequencing information indicating the order of the packet with respect to other packets sent at different times. The packet may include a tail portion, which may indicate the end of the packet.

In some embodiments the payload may be further expanded using error correcting coding methods. One such coding method is repeat each bit number of times. For example, if each bit is replicated 3 times, it is possible to detect a single bit error by majority logic. More powerful error correcting codes are also well known, such as, such as forward error correction (FEC) codes, including BCH codes, Reed-Solomon codes, convolutional codes and others may be used in at least some embodiments described herein. If the message elements are pre-stored in memory the use of more complicated codes than the repetition code just described can be straight-forward since the encoding may be done in fabrication of the transponder memory (that is, the message code may be hard coded). In either case it will be clear to one of ordinary skill in the art that that there are many coding schemes which may be used. These codes are also able to detect error conditions. This is a form of conditional transaction that causes the interpretation of the feedback information to be modified. For example, if the feedback information payload is received with recipient address identification code deemed intact, per the FEC check process, but the event code damaged or in error, the operator of the campaign may still infer that the parcel was opened with a calculable probability, rather than damaged by fire, for example. There are many statistically meaningful information extraction possibilities associated with receipt by the terminal processor of a partially correct feedback information payload. Another example is that the address code is errored, but the event code is intact. Then the operator may still count the parcel as “opened” in calculating the overall “open rate,” which is one statistic provided by the techniques of the present disclosure.

In the radio protocols employed in LP-WAN there is raw capacity (allocated bits of message payload) available beyond that required to report the data of interest. This capacity (or bits) may be utilized to add redundancy to the data in a manner that permits either or both of the transmit power and or the transmitted energy to be reduced. The energy reduction comes about from utilizing coding methods that reduce the energy per bit of information needed for reliable transmission. It is noted that the number of information bits is predefined by the use case or application. The number of code bits transmitted may be varied within the allowable payload size parameters. By including code bits, albeit additionally to the information bits, the net energy expended per information bit may be reduced. This reduces battery size and peak current. Another way in which peak current, which may be more important to minimize than energy, may be reduced is to reduce peak transmitter power levels. There are two ways to reduce peak power. The first is to simply distribute energy over a larger number of bits. Power is energy divided by time, so this larger spread of energy reduces power requirements even if the energy is the same. From this perspective, the energy of the entire message should be distributed over the largest time so that power is minimized. The other power reduction aspect is direct: reducing the energy reduces the power since the time available to transmit the message is fixed and bounded. Energy reduction using codes will achieve power reduction.

Another aspect of power and energy allocation pertains to the overhead needed to establish a wireless connection. These overhead functions include synchronization functions that take place prior to information transmission or reception. It can be desirable to balance the synchronization function with the information transmission function. If the information transmission function is optimized to minimize energy or power while the synchronization function is not similarly optimized, or at least balanced, the excessive power and energy consumed by synchronization can spoil the advantage gained in the information transmission aspect. Thus, the transmitted waveform may be optimized in a manner that simultaneously reduces the peak current demanded for information transmission and synchronization.

Once the composite binary signal is prepared, the modulation process may be applied to it to create a radio frequency signal. The modulation process may be specific to the radio network employed. There are numerous modulation processes to choose from. Any modulation process can be used that is compatible with the wireless network 220.

In some embodiments, the tracking device 100 may send repeated transmissions of the status information to help ensure it is received by the network 200. To do so, the processor 110 may be configured to transmit the radio signal repeatedly. For example, repeated transmissions of the same packet may be performed. The tracking device may send a single transmission with the status information or any number of repeated transmissions of the same status information, such as between two and ten transmissions, or more. In some embodiments, the processor 110 may be programmed or otherwise configured to stagger the repetitions of the radio signal transmission so as to minimize burst traffic overloads on the wireless network.

### Fabrication Techniques

One particular aspect of the present disclosure is the use of “printed electronics” to create the electrical circuitry on the tracking device 100. Printed electronics permits the tracking device 100 be lightweight and flexible. By printing circuitry, battery and antenna elements using a single process, or a small set of processes arranged to create desired characteristics, the tracking devices may be produced in large quantities using automated high-volume manufacturing equipment.

There are several ways to produce the circuitry with components as functional modules. In one embodiment, one may use a printed circuit board (“PCB”) made of an epoxy with a copper layer, having a protection layer on the copper, whereby a portion of the protection layer is removed. In this embodiment, the free copper area etched. The rest of the protection layer may be stripped from the remaining copper traces. One may also perform stencil printing of solder paste to the copper traces. Additional components such as semiconductor packages and/or discrete components may be attached in any suitable way, such as “pick & place.” Such components may secured to the PCB via a heating process in a reflow oven for soldering. In an alternate embodiment, the circuitry may be produced by using a rigid epoxy-copper PCB a thin and flexible Polyimide film with a copper layer in place of the PCB.

In some embodiments, the circuit could be printed via a printing process for conductive traces (e.g. silver traces) to a film (e.g. Polyethylene terephthalate, Polyethylene naphthalate, and the like) using stencil printing of solder paste or normal printing of a glue or binder, which conductively glues the components to the silver traces. In this embodiment, it may be advisable to use low temperature solder paste because these polymer films cannot stand the high solder temperatures of normal soldering. In this embodiment, the circuit could be cured at room temperature or heated up for faster curing. This embodiment could also place components via “pick & place,” followed by heating up for soldering.

The printing methods for printing the circuits can vary in alternate embodiments. In one embodiment, one could use a screen printing method or process, which has the advantage of providing relatively thick conductive layers with sufficient resolution. In an alternate embodiment, one could use a gravure printing process, which has the advantage of having a lower thickness than screen printing, while still having good resolution.

The printing aspect of the present disclosure includes the use of either conductive ink or metal deposition or a hybrid arrangement according to the conductivity required in a given portion of the circuit.

In an alternate embodiment, the circuit could be printed with a flexographic printing method, which provides thickness similar to gravure and sufficient resolution.

In alternate embodiments, in order to increase thickness, any of the above printing methods could be combined with printing seed layers having electroplating or electro-less platting thereon.

In these methods, diverse curing methods, such as convection heating, infra-red, ultraviolet, ultrasonic, and photonic, could be used. These curing methods are also useful if sintered nanoparticles are used.

Many different materials can be used to print conductive traces, including without limitation: carbon (including graphene), silver, copper, silver plated copper, organic conductive polymers, tin, and inorganic materials like indium tin oxide.

Since the devices described in the present application may be extremely lightweight and physically small, and moreover in certain applications disposable, it may be desirable to have a fabrication process that is consistent with these physical characteristics while at the same time being amenable to high speed production at low cost. There are a variety of materials available for use as substrate, ranging from paper to conventional printed circuit boards (PCBs). In addition, the layout and method and interconnection of the components plays a role in determining the cost and production rates achievable. A material that is intermediate in rigidity, flexibility, weight, strength and durability between paper and PCB is Polyethylene terephthalate (PET). This material is durable and inexpensive and is well suited to applications in which the tracking device is desired to be low profile (flat) or conformal (within the packaging containing the item being tracked or monitored).

The challenges faced in fabrication include the following:
1. Electrical connections via soldering require heat that can damage certain substrates, including PET.
2. Connections established should not become loose or break.
3. Conductive glue can difficult to apply to very finely featured parts, such as integrated circuit chips with small or finely pitched pins.
4. Flexible substrates such as PET are subject to warping or other distortions as the tag passes through an automated high speed fabrication machine or process. Such distortion is further exacerbated by heat, as in soldering.
5. Printed electrical circuitry may incorporate sufficiently conductive ink, which translates into a sufficiently thick deposition, while at the same time being able to be cured (dried) rapidly enough to maintain a production flow rate.

A fabrication method that solves these problems is described next. As will be clear, the following description is by way of example and is not intended to be limiting.

Low Temperature Soldering

Low temperature soldering is using solder pastes often consisting of Sn/Bi (tin/bismuth) combinations, also including a flux agent. These can have melting points of approximately 140° C. That allows soldering temperatures between 150 and 200° C. Such solder pastes are used either for energy saving soldering, because of the lower temperatures or used because the used substrates or components would be damaged by the high temperatures applied through conventional soldering. In reflow-ovens a temperature of 160° C. has been found to work well.

Carrier System

In pick&place and soldering lines PCBs are the substrates to be equipped with chips and components. PCBs are normally of sufficient thickness to be transported in a transport system consisting of two tiny transport belts. Two edges of the PCB are touching these belts. The middle part of the PCB is not supported. Since PET films have not enough stability to be transported in such a pick&place and soldering line, carriers may to be used to give the substrate enough stability to be transported. The carrier can be a more rigid material to which the substrate is temporarily glued with non-permanent glue. Once the substrate is secured to the carrier the soldering process may be applied. Spray glue has been seen to be effective, and silicone based glues offer greater elasticity and heat resistance. After the solder sets the substrate may be subsequently separated from the carrier.

A variation of the above method is to apply the bonding agent (glue, tape, etc.) locally to the substrate, in particular, under the sites of attachment of components, leaving other portions of the substrate material somewhat more free to expand and contract in response to the stress of the soldering heat.

To apply the soldering heat the substrate is passed over a hot plate. This is a more compact and energy efficient method than using a reflow oven.

Customized Hot Plate

The plate may be customized so that the “hot spots” are precisely located to achieve successful solder flow without introducing heat to a large area. This (method, approach, procedure, design) minimizes the distortion of the substrate. Ideally the plate should have high heat locations that are matched to the solver bond locations and as small as possible.

Benefits of the Above Process/Method/Procedure
1. Greater precision of heat control, both location of heat application and temperature
2. Protection of the substrate from distortion
3. In particular, protection of the soldered connections from breakage induced by substrate expansion and contraction in the vicinity of said solder connection
4. Higher speed of production due to less total heating and therefore more rapid cooling

### Example Applications

Some applications of the present technology relate to tracking the status of mailings or parcels. Previously, the Media Mail-based marketing and advertising business has provided only the most primitive feedback relating to the effectiveness of a given mass mailing campaign, namely, whether or not the person converted (the definition by which varies from advertiser to advertiser), and only in such events, the mailing campaign operator (or other surrogate agent) will receive positive notification of said recipient's interest in and response to the material. However, in the vast majority of cases, the media or parcel is discarded unopened, opened and then discarded, destroyed by the recipient (e.g., shredded) or allowed to remain in any one of a number of states of oblivion.

In profound contrast, in the electronic commerce and digital marketing/advertising paradigm, which is enabled by the Internet and by the concomitant profusion of personal digital electronic technology such as smart phones and wireless computer tablets, operators of digital and electronic advertising and marketing campaigns are afforded the powerful benefit of specific feedback from the recipient in the form of tracking, logging, parsing and reporting recipient's actions through the use of “cookies”, pixel tracking, and various other means. These actions include “clicking” on content, reading content, saving content, and other information that can be subjected to data analytics that reveals recipient's reaction, interest, intent and susceptibility to the marketing proffer. It is estimated that the use of these feedback techniques increases the effectiveness and efficiency (cost/acquisition of a customer) of the marketing or advertising campaign by 300 to 500 percent.

In view of the inherently blind, open-loop (no or limited feedback) nature of the conventional non-electronic mode of marketing, and in view of the immensely more effective and more efficient electronic (Internet and mobile phone based) alternative, the inventors have recognized a need to improve upon the methodology of conventional mail marketing operations that rely on consumption by the recipient of hard copy items. The inventors have appreciated it is desirable to measure the rate of consumption of the media and other forms of disposition of the media.

The present disclosure provides a low cost information feedback system that can report a recipient's actions and reactions to the marketing campaign operator automatically, in real time. In particular, in one embodiment, a method for determining if a marketing parcel has been opened is provided. It will be clear to those familiar with marketing analytics that there are additional types of information that may be provided in the feedback message, including time stamps of events (delivery, opening, etc.). In applications in which the response of a recipient of a marketing or advertising offer or related material is of primary interest, the present disclosure can provide behavioral information about the recipient. Furthermore, the present disclosure may do so in real time, sending the feedback message immediately in response to the item being handled (opened, read, torn, etc.) by the recipient.

In one exemplary use case, the tracking network 200 could be used to determine the outcome of a mailed marketing campaign. The tracking device 100 may be embedded within or attached to the packaging, wrapper or envelope carrying the targeted recipient materials (marketing offers, surveys, subscriptions, giveaways, advertising, goods, etc.). In certain applications, recipient action upon taking delivery, either actively or passively, of the parcel is sensed by the tracking device 100. Actions may include no action (the parcel is never opened), open and discard, open and examine/read the contents, shred or tear, burn, or any other behavior that may be measured by sensor(s).

In this use case, once the parcel events, meaning the events that occur with respect to the parcel 210 at an individual mail recipient's home, have been deemed to have run their course, the next step could be transmitting an event code from the tracking device 100 to the network 220. The event codes could reflect parcel events such as whether or not the parcel 210 was opened. If the parcel was never opened, in some embodiments, there may be no signal sent over the tracking network 200. In other embodiments, a system timeout could be employed whereby an event code is transmitted after expiration of the timeout period indicating that the mail recipient never opened the parcel 210, which presumably means it was discarded.

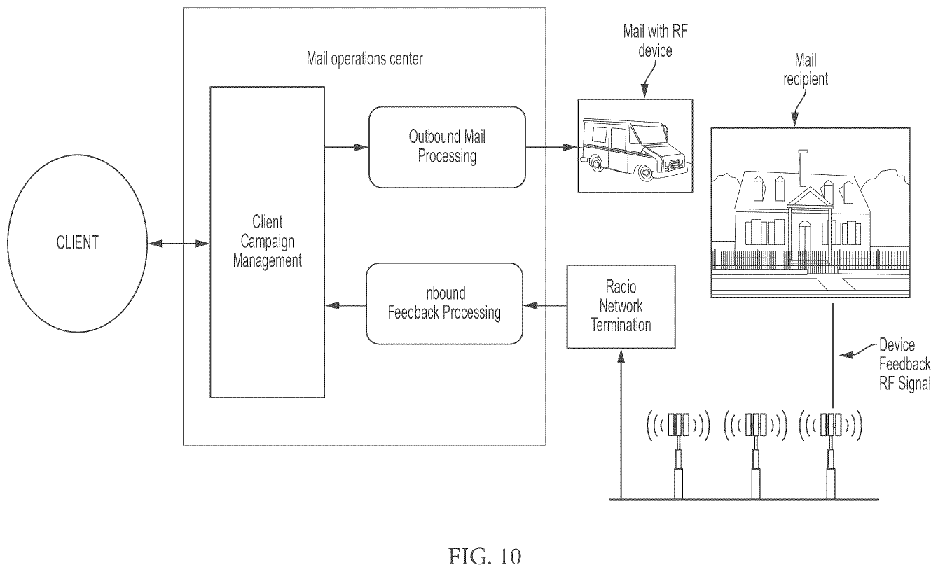

FIG. 10 illustrates an exemplary system for tracking the status of mailings. A mail operations center may process outgoing mail, which may then be delivered by any suitable delivery service, including but not limited to the U.S. Postal Service. The mailings may then be delivered to recipients. The status of the mailing, such as whether or not a parcel was opened, is then transmitted via wireless network 220. Computing device 230 may receive the information and be used to perform inbound feedback processing and client campaign management.

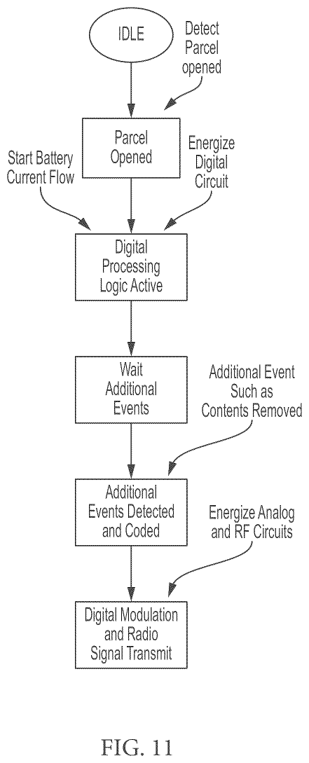

FIG. 11 shows a flow chart for the intelligent transponder operation when the system is configured to detect “parcel opened.” A sensor 170 may include a conduction path that is opened or closed when the parcel is opened. The conduction pay may include fine wire or printed conducting material. The sensor 170 may detect whether the conduction path is opened or closed using any suitable technique, such as detecting the voltage and/or current at one or more terminals. In some embodiments, detecting that the parcel is opened may trigger turning on power to one or more portions of circuit 10, such as processor 110 and/or transmitter 120. In some embodiments, when the conduction path changes state, the tracking device 100 is triggered to transmit a signal including information indicating the updated status. The tracking device may or may not wait for additional events to occur, such as detection by a sensor 170 that the contents of the parcel have been removed. In other words, the transmission of information may be triggered either by the detection of the parcel being opened or the detection that the contents have been removed. The information indicating the detected status, such as a parcel being opened and/or or contents being removed, may then be encoded, modulated and transmitted.

Various sensor arrangements may be used to detect the status of a parcel, such as whether the parcel has been opened and/or the contents removed. An example of an arrangement is to allow the sensor circuit to support a very small current when the parcel is undisturbed. Upon opening the parcel this current will be interrupted, for example, by breaking a very fine wire. In this embodiment a small battery discharge due to the small sensor circuit current flow prior to occurrence of “parcel opened” may be acceptable. However, the continuous flow of current may drain the battery, reducing its lifetime and potentially preventing successful transmission of the status information. However, such an arrangement can be utilized if such current is not excessive.

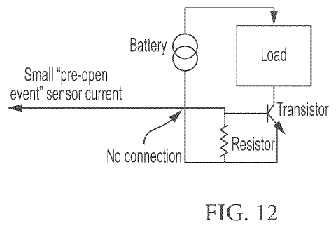

In the case of the sensor circuit operation just described, in which a small current flows in the sensor loop, a way of controlling the battery current flow to the rest of the circuit 10 or a portion thereof may be implemented. This may be accomplished by utilizing an active switching element, such as a transistor. FIG. 12 shows an example of a control circuit that can implement such a control technique. Ancillary circuit elements, such as resistors and possibly capacitors, to optimize the circuit behavior and minimize dissipation may be provided.

In FIG. 12, the Load is a circuit that is not energized until the sensor current is interrupted. A small sensor current flows through the resistor and the sensor (at the left) as long as the package is not opened (the sensor carrying the current is not broken). As a result of this current, a voltage is established across the resistor and the transistor is “turned off” (non-conductive) because the base-emitter junction is “back biased.” When the sensor current is interrupted, current stops flowing through the resistor, which changes the voltage at the base of the transistor. The transistor “turns on” and the load draws current from the battery because there is now a complete current path through the load and the battery. While the transistor is in the off state the load cannot draw current since there is no path for the current through the Load.

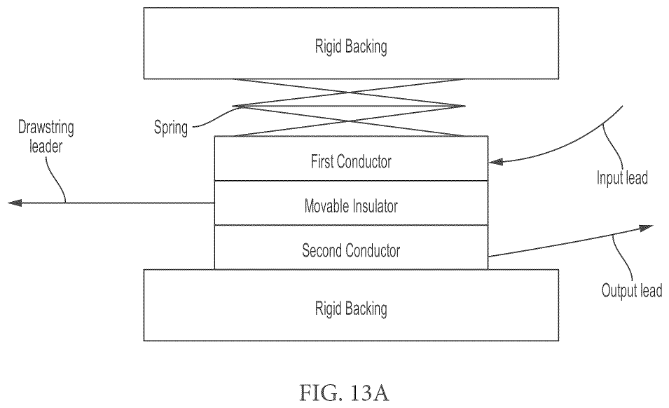

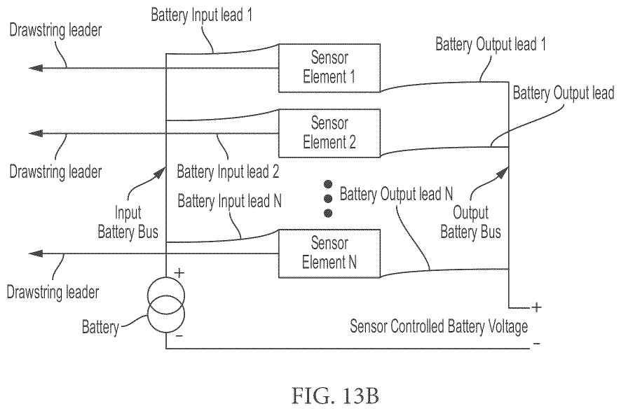

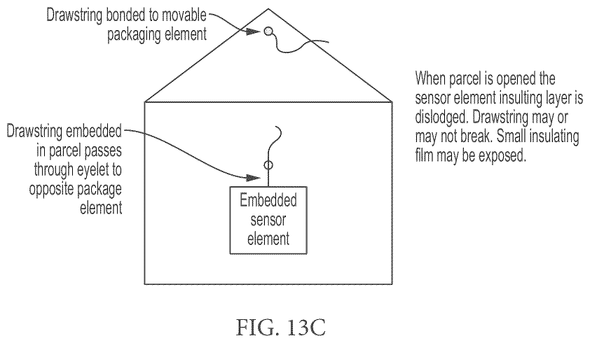

In order to minimize current drawn prior to the “parcel opened” event an alternative approach may be adopted. In the alternative embodiment the sensor subsystem is designed to operate in the following manner. The first level sensor is again designed to detect and identify the “package opened” event. In one embodiment, the sensor arrangement may be such that the transponder circuitry is in a quiescent state, drawing minimal or no power, or, in case of a clock or timer element being included, that the clock be the only energy drawing element prior to the “package open” event. This is not intended to be limiting, but rather descriptive of some embodiments. Thus, the first level sensor may operate by closing a circuit when the parcel is opened. One method for arranging this is shown in FIGS. 13A and 13B

FIG. 13A shows an arrangement of an exemplary sensor 170 including two conducting sheets separated by an insulating sheet in a “sandwich” arrangement. The sandwich arrangement is kept under pressure by a spring or other device that produces a spring force. The drawstring leader may be a strong string, such as fine fishing line, that is connected mechanically to the parts of the packaging that are expected to be torn when the parcel is opened. One can envision multiple configurations in which opening the parcel causes the drawstring to be pulled. One such example is shown in FIG. 13C. When the drawstring is pulled the moveable insulating layer of FIG. 13A slides out from between the conductors allowing contact between them, and thereby allowing battery current to flow. For parcels in which it is envisioned that multiple possible opening actions exist one may utilize a parallel combination of sensor elements as illustrated in FIG. 13B. In FIG. 13B, when any one or more of the sensor elements is closed by the action of the drawstring, the battery circuit will be closed and battery voltage will be available for use by the transponder circuitry.

Optionally, additional sensor circuits may be included that can detect the removal of the contents from the packaging. For example, a connection to the packaging is provided between the contents and the packaging so that the removal of the contents, is detectable. Such a connection can be provided via thin conducting film, foil or similar material bonded to the inside of the packaging envelope and, separately, to the contents, for the example. When the contents are in the envelope the circuit is closed and current flows. When it is removed, the circuit is broken, which may be detected. However, as discussed above, other techniques may be used that detect closing of a circuit rather than opening of a circuit, which can reduce power consumption

Some embodiments, there is a conditionally executed process involving the second sensor: it only functions if the parcel is opened. It may not be necessary to determine that the contents were not removed if the parcel is not opened. Upon detection of the “parcel opened” event the flow of current creates a voltage across a sensing resistor shown in FIG. 12, that in turn wakes up the digital processing circuitry.

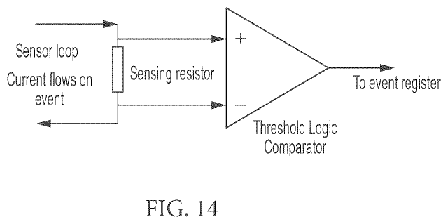

There are other triggers and events that may be identified by the sensor(s) 170. The various sensor circuits, and the events they are designed to detect and identify, may produce a variety of currents and voltages that may be detected and analyzed. There are numerous electronic circuits for sensing such voltage and currents, one of which is illustrated in FIG. 14. FIG. 14 illustrates that a sensing loop may include a sensing resistor. When current flows in response to an event, the current produces a voltage across the resistor. The terminals of the resistor may be connected to the inverting and non-inverting inputs of a comparator. The comparator may detect whether the voltage across the sensing resistor is above or below the threshold, and produces an output indicating the comparison result.

Another way to detect different events is via multiple I/O terminal(s) 141, which are connected to different sensors. Thus the events would be I/O mapped. Alternatively or additionally, the events may be mapped into interrupt vectors.

When the digital processor is activated by the “parcel opened” event the information detected by the sensor may be used to construct a pointer that instructs the processor 110 how to assemble a message. This instruction can be processed either as a program jump in a software implementation or as a pointer (address) to a memory location from which the appropriately coded message element is retrieved. The latter method has the benefit of using low cost memory circuits rather than requiring a processor to build the element “on the fly” from primitive instructions. In case the system is configured to detect the “contents removed” event, the processor may enter a “wait” state so that the second event may be detected before the final message element is selected or assembled.

The status information may be transmitted by the tracking device 100 at a variety of times. One example is transmitting the information in response to detecting a change in the status information. Another example is transmitting the information once a time period has expired. The clock 160 may be used to set a suitable time period. In some embodiments, the time period may be set to expire at a point after a recipient likely would have opened the parcel. If no “parcel open” event has been detected, the tracking device 100 may then send status information indicating that the parcel has not been opened prior to expiration of the time period. Alternatively, the tracking device 100 may be configured not to transmit any information unless a suitable event occurs. In such a case, it may be decided that a parcel has not been opened, or the contents not removed, if the computing device 230 has not received information from the tracking device 100 within a given time period.

In addition to the above use case, the present disclosure enables applications to a plethora of information gathering, item tracking and behavioral analytic applications. In addition to mailings as described above, the use of the present disclosure in a magazine or similar encapsulation provides an effective and highly efficient method for “closing the loop” on advertising content delivery through advertisements in said magazines. By incorporating sensors within the advertisement itself, or multi-sensors within a plurality of advertisements, advertisers may obtain crucial consumer feedback. The types of feedback and the activation of the feedback by various stimuli (scratch off material, peel off member (e.g., sticker), pressure sensor or other tactile input mechanism) are numerous. In this type of application the need for microminiaturization may be lessened due to the form factor and weight of the carrying media (magazines, for example).

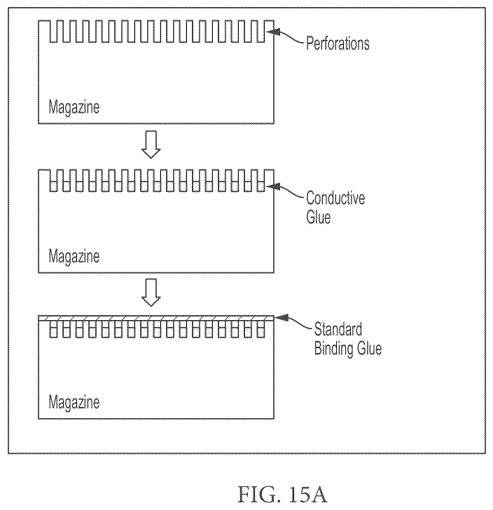

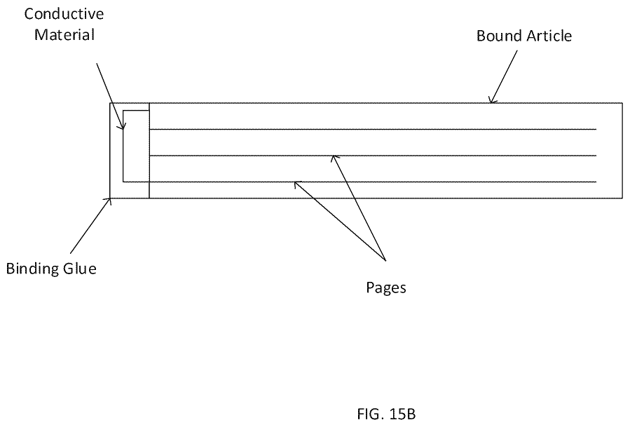

FIG. 15A shows an example of a process that can provide a conductive connection through one or more individual pages of a magazine or other bound article. As illustrated in FIG. 15A, perforations may be formed in the pages where they are to be bound together. As illustrated in FIG. 15A, the perforations may be notches in the pages. A conductive material such as a conductive glue may be applied, and may be disposed within the perforations. The conductive material may be applied in any suitable way, such as dipping, for example. The conductive material may partially fill the perforations, as shown in FIG. 15A, or may completely fill the perforations. After applying the conductive material, the article may then be bound by applying a binding glue to bind the pages at the perforations. The binding glue may be a standard binding glue. As a result, the conductive material extends through a plurality of pages of the bound article, and can be used as a “via” for making conductive connections between different pages. FIG. 15B shows a cross-section of the conductive article, illustrating that the conductive material in a perforation extends through the thickness of the bound article and contacts a plurality of different pages. Accordingly, the conductive material can make conductive connections to conductors printed or otherwise disposed on various pages. In some embodiments, the conductive material in each perforation may serve as a separate connection that is electrically isolated from the conductive material in other perforations. This allows making a plurality of connections through the thickness of the bound article.

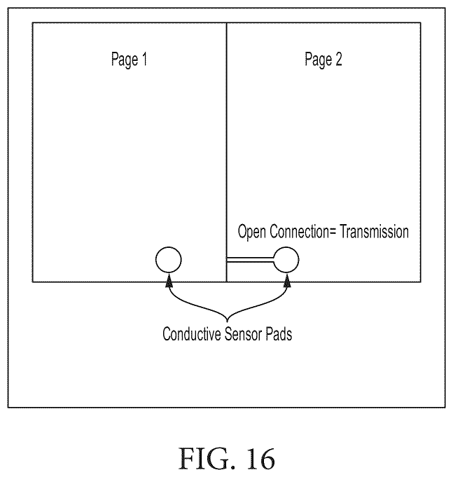

FIG. 16 shows an example of a sensor that can detect opening of an item. In this example, the sensor can detect opening of a magazine or other bound article at a particular page. Conductive sensor pads may be printed on two facing pages such that they are in physical and electrical contact with one another when one page is closed on the other (e.g., the magazine is not open to the page). When the bound article is opened to the page, the conductive sensor pads are no longer in electrical contact with one another, and the sensor detects an open circuit. In response to detecting the open circuit, a tracking device embedded in or attached to the bound article may transmit status information indicating that the page has been viewed. Alternatively, the information may be transmitted at a later time. This information may allow evaluating the reader's consumption of the bound volume, such as determining which portions of the bound volume, such as articles or advertisements, have been viewed.

The present disclosure permits affirmative consumer actions to be tracked. Examples of such are transaction processing via “peel/click here to learn more,” and “peel/click here to buy” processing. One difference is that the detection of the opening of a package or removing contents is passive, and does not require the recipient to take any action beyond the action recipient would ordinarily take. The possibility of accepting “active” stimuli rather than only passive, such as by providing an electrically enabled “peel here” label or similar means, provides for additional applications. By incorporating sensors within an advertisement, or multi-sensors within a plurality of advertisements, advertisers may obtain crucial consumer feedback.

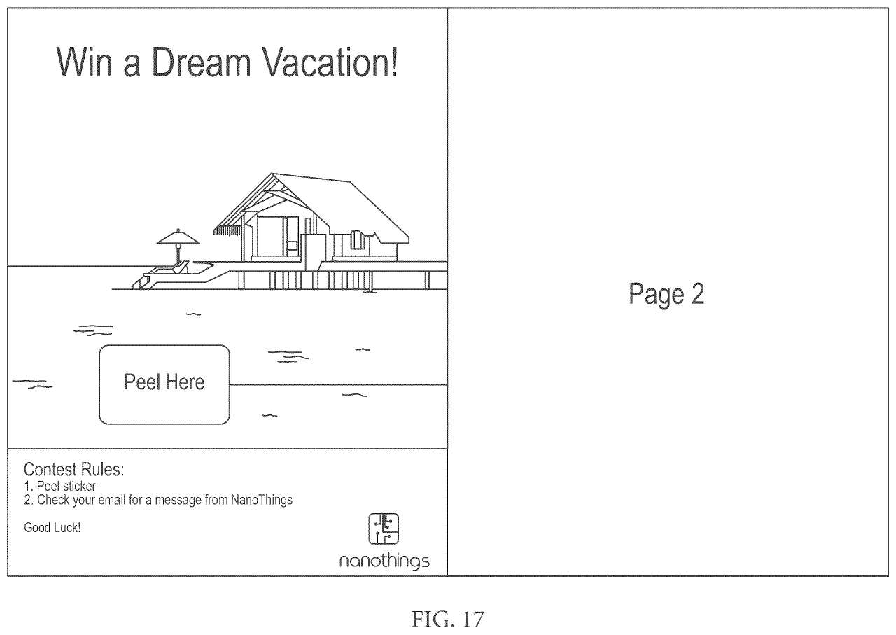

FIG. 17 shows an interactive sensor. Such a sensor may be included in a printed advertisement, such as a magazine advertisement, for example. In the example shown in FIG. 17, the advertisement indicates that the reader can enter a contest to win a dream vacation. The reader is instructed to peel a sticker. The sticker may have a conductive backing that provides electrical continuity within a sensor circuit when it is in place. When the sticker is peeled or otherwise removed, the continuity in the sensor circuit is broken, and the sensor detects an open circuit. In response to detecting the open circuit, a tracking device embedded in or attached to the bound article may transmit status information indicating that the sticker has been removed. Alternatively, the information may be transmitted at a later time. As discussed above, the status information indicating the sticker has been removed along with identification information of the tracking device is then transmitted by the tracking device 100 through the wireless network 220 to the computing device 230. After receiving the status information and the identifier, the computing device 230 may look up contact information for the recipient of the advertisement. For example, the computing device 230 may access a database that maps the received identifier to the recipient's mailing address, e-mail address or phone number. A communication may then be send to the recipient indicating that they have been entered into the contest and/or requesting further information to enter the contest. Such an indication can be send in any suitable way, such as by e-mail message, text message, telephone call, or by mail. Other communication(s) may be sent to the recipient indicating whether they have won the contest, for example.

Although the example has been given of entering a contest, there are many other applications of an interactive sensor. For example, removing a sticker associated with an advertisement may trigger sending a coupon to the recipient. As another example, removing a sticker may trigger ordering an item, which may then be delivered to the recipient either in physical form or virtual form. As another example, removing a sticker may be an indication of a request for further information, and in response to receiving this indication a representative may contact the recipient of the advertisement for follow-up. As another example, an interactive sensor may be used to track delivery of an article. For example, a sticker may be removed when an article is delivered to a recipient, which may allow tracking that delivery has occurred and/or the time of delivery.

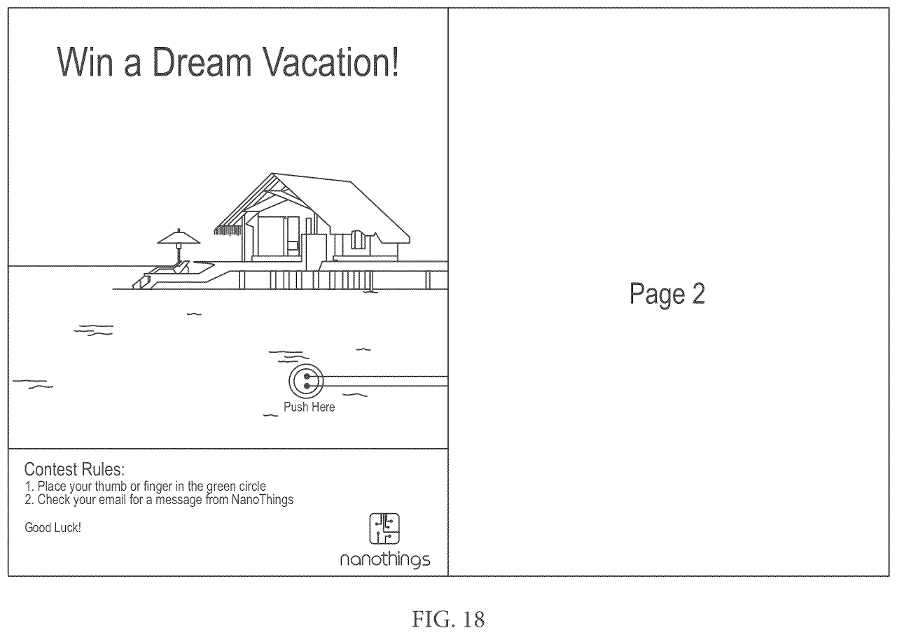

Although an example has been described in which a sensor detects peeling of a sticker, it should be appreciated there are many other ways that interaction with an article can be detected. FIG. 18 shows an example of a contact sensor that includes two conductive regions separated by a distance smaller than the width of a finger. The reader may be instructed to push or touch the two conductive regions at the same time. A finger contacting the two conductive regions may be detected either by a reduction in the electrical impedance across the terminals or by a change in the capacitance between them.

There are also other ways that interaction with an article can be detected. For example, an instruction may be provided to scratch off a conductive region, which may break electrical continuity. Another example would be instructing the user to break a portion of the conductive path, such as by tearing off a portion of the page that includes a portion of the conductive sensor path.

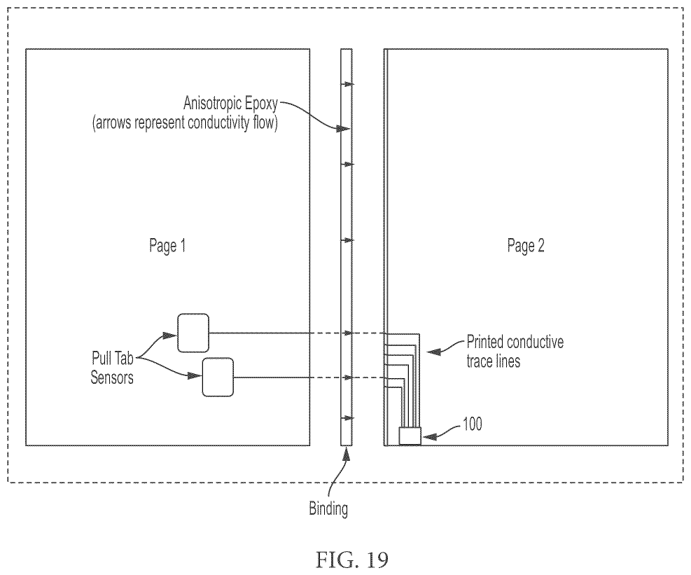

FIG. 19 shows another way of providing electrical contact to one or more individual pages of a bound article, such as a magazine, for example. In this example, the binding glue may be a conductive material, such as an anisotropic epoxy that allows current to flow in one direction. One or more sensors such as pull-tab sensors may be disposed on a page, with conductive traces extending between the sensors and the binding glue. The tracking device 100 may be formed on another page with traces extending between the tracking device 100 and the binding glue. In this example, the binding clue conducts anisotropically such that it allows current to flow in the horizontal dimension of FIG. 19 but not the vertical dimension. This allows distinct conductive connections between the sensors and the tracking device 100 through the binding glue.

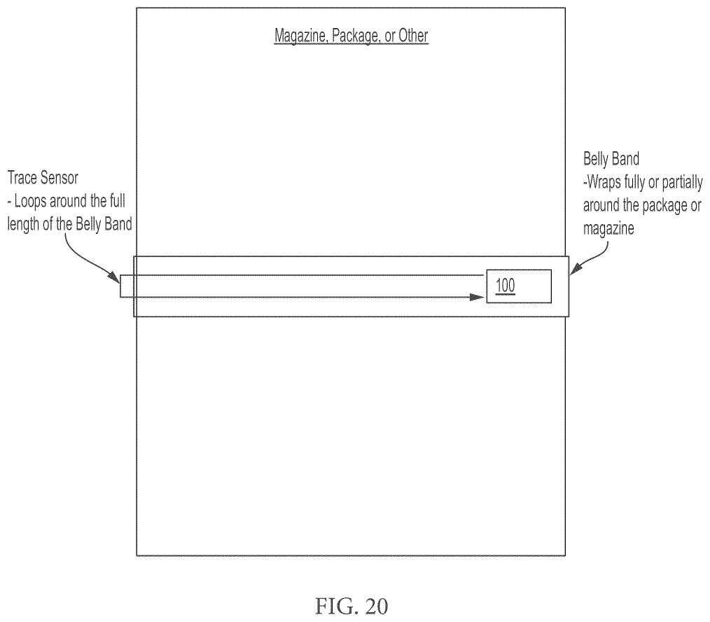

FIG. 20 shows another method for providing the technology, in a magazine application for example, is to utilize “belly bands.” A “belly band” is a band that may be wrapped around a magazine or package. One version of a belly band could be a thin strip of paper wrapped around the exterior of a magazine or package that can accept a wrapper. A belly band may include the tracking device 100, antenna and one or more sensors 170 to detect removal or opening. The band can easily be added to pre-produced media such as magazine as well as to other packaging that can accept a stretch wrapper. The sensors within the device can detect various actions, such as removal or opening of the wrapper, which may be done in the ways discussed herein. Additional sensor leads may also be provided so that internal or external sensor action can be connected to the transponder.

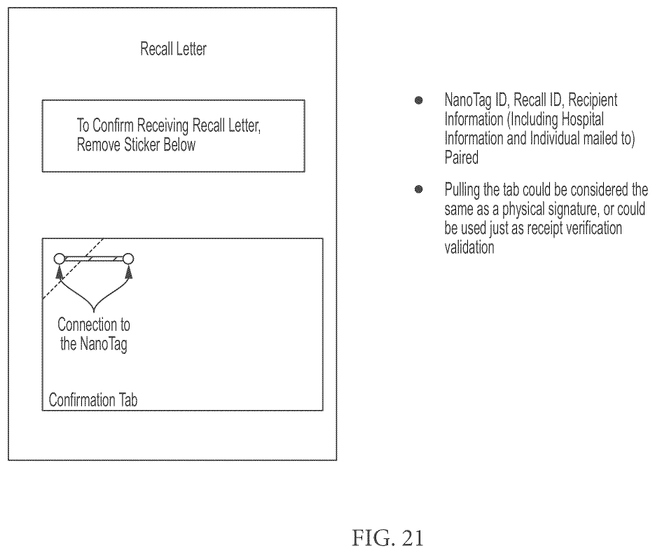

FIG. 21 shows an example in which a sensor may be used to provide confirmation that a parcel or other article has been received. For example, it may be used to confirm whether a recall letter has been received. The recall may be for any type of item, such as medical devices, food, medicine, an automobile, or a child safety seat or toy, by way of example. One application in the medical field is to medical device recalls. In certain cases there is a mandatory procedure for compliance with recall protocols such as may be instituted by the Food and Drug Administration (FDA). Use of the present disclosure provides verifiable compliance checking and can enhance the safety intended by any such recall. The disclosed devices can be embedded into or attached to recall letters. When the recipient pulls a confirmation tab, as illustrated in FIG. 21, or when the letter is opened as discussed above, an event code can be transmitted back to computing device 230. The event code would provide verification of receipt of the recall notice, which would be helpful compliance data for the recalling company or entity to obtain. As with FIG. 17, in some embodiments pulling the confirmation tab may remove a conductive sticker that breaks electrical continuity, which can be sensed as discussed above. However, any of a variety of sensing techniques may be used, such as those disclosed in other figures, such as FIG. 18, for example.

Parcel tracking can be implemented directly by including a tracking device in the packaging of the parcel. Parcel tracking can be used to monitor the progress of the parcel through delivery channels. Alternatively or additionally, by configuring the tracking device with appropriate sensors, the conditions to which the parcel is subjected in transit may be monitored and reported. These conditions include temperature, humidity and/or other ambient environmental conditions. Other conditions that may be sensed include conditions that may be harmful to the parcel, such as smoke, fire, harmful gas, loss of air pressure, loss of oxygen, etc. For example, a temperature sensor may trigger transmission of a radio signal including status information indicating that a temperature is outside of a safe range. In this event the tracking device 100 is again awakened by the sensor produced current flow and, as in the other illustrative cases, assembles or retrieves the appropriate feedback information message element and embeds it in the radio signal payload.

However, this is by example and not limitation, as it will be clear to one of ordinary skill that other variables may also be monitored. For example, gravitational force (acceleration, or g-force) may be monitored for particular applications. Other non-limiting examples include measuring of relative humidity, a traveling velocity, an acceleration, a measure of light, a shock, a pressure, a vibration, a location, a gas, a fire, an orientation in space, a g-force, a sound, a stacking height, a weight, or a state of integrity. The present disclosure provides the capability for real time automatic monitoring and reporting of environmental parameters. It is also possible to monitor the absolute orientation of an item in transit in case it is desirable to maintain a particular orientation, such as “this end up.” Packaging can also be outfitted with the present disclosure configured to operate in a manner that indicates the loading of items in situations where “stacking” limits are in place (such as “do not stack more than 10 high”).

Another application of the techniques described herein is asset tracking. Primary assets may be tracked in a manner similar to the tracking of parcels. Certain secondary assets are used in the warehousing and shipping of primary assets. An example of a secondary asset is a pallet. Pallets are used in transport and warehousing operations in which primary assets are loaded on pallets for movement or storage. The pallets are intended to be reusable. Pallets are not generally the end-user item that is being transported to a given location, and they are usually expected to be returned to their owner. A problem that arises in the flow of pallets through a logistics process or channel is that they are often lost or discarded when the primary assets are unloaded. The present disclosure provides a means to track the location of pallets through their movements and even throughout their useful service life. By integrating a tracking device into the pallet or other secondary asset as described above, or attaching the tracking device to the asset, the asset can be tracked, and the likelihood of an asset reaching its proper destination can be improved. For example, the likelihood of pallets being returned to their owner can be increased. In some embodiments, larger assets such as pallets may allow the tracking device to be powered by a larger battery and/or by a renewable power source such as a photo-cell or solar-cell. The asset may be tracked on a regular basis, such as hourly, daily or at any other suitable interval, or in response to detection of a condition by one or more sensors.

The present disclosure usage can be extended to other areas such as delivery services. One such application is perishable item delivery, such as food or medicine. The present disclosure can also be generalized to provide detailed tracking capabilities to parcels rather than merely delivery event reports. In addition, tracking of items other than mail items can be considered, including animals, people, automobiles, and even personal items such as purses, wallets, a package, a product, a container, a pallet, timber, a piano, a case of wine, blood or other health-related materials, a vehicle, a pet, a human, an electronic device, frozen food, perishable items, and so forth.

In the medical field the present disclosure has utility as a tracker of medical materials. Examples of medical materials include perishable materials such as medicines and live tissue or organs. Other examples include blood, urine, biopsies, cryogenically frozen materials, humans, cadavers, and the like. The vitality of such items depends critically on the timeliness of delivery as well as maintaining a controlled environment. Use of the present disclosure provides a method for monitoring and validating these variables.

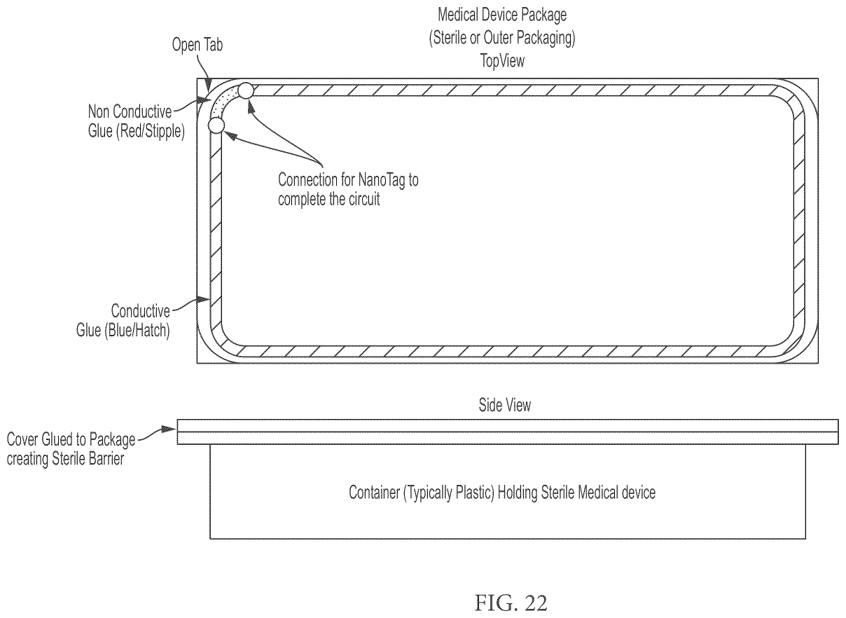

FIG. 22 shows an example of using a tracking device to monitor the integrity or opening of a medical device package. A medical device package may be a sterile, sealed package. Electrical conductors forming an electrically conductive path may be disposed on or in the packaging material such that the electrically conductive path is broken when the package is opened. For example, in the embodiment of FIG. 22, a medical device package may be opened by pulling on an open tab to remove the cover of the package. When the open tab is pulled, the electrically conductive material shown in the upper left of FIG. 22 is separated from the electrically conductive path. The opening of the package can be detected and reported by the tracking device 100. This may serve as an indication that the medical device has been used, and may allow automated inventory tracking. Accordingly, a hospital or other facility can more easily track its inventory and can be informed as to whether inventory should be re-ordered manually. Optionally, the re-ordering may be automatically performed when supplies fall below a certain level.

### Additional Aspects

As discussed above, the functions described herein may be controlled by one or more controllers. Such controllers may be implemented by circuitry such as electronic circuits or a programmed processor (i.e., a computing device), such as a microprocessor, or any combination thereof.

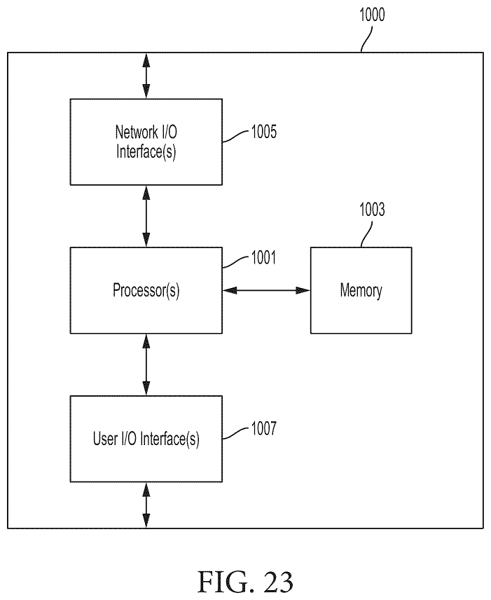

FIG. 23 is a block diagram of an illustrative computing device 1000 that may be used to implement any of the above-described techniques. Computing device 1000 may include one or more processors 1001 and one or more tangible, non-transitory computer-readable storage media (e.g., memory 1003). Memory 1003 may store, in a tangible non-transitory computer-recordable medium, computer program instructions that, when executed, implement any of the above-described functionality. Processor(s) 1001 may be coupled to memory 1003 and may execute such computer program instructions to cause the functionality to be realized and performed.

Computing device 1000 may also include a network input/output (I/O) interface 1005 via which the computing device may communicate with other computing devices (e.g., over a network), and may also include one or more user I/O interfaces 1007, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

Various aspects of the apparatus and techniques described herein may be used alone, in combination, or in a variety of arrangements not specifically discussed in the embodiments described in the foregoing description and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

1.-89. (canceled)

90. A tracking device for use in a Low Power Wide Area Network (LPWAN), comprising:
a printed antenna;
an energy storage device;
a transmitter powered by the energy storage device; and
control circuitry configured to control the transmitter to transmit, over the LPWAN, using the printed antenna, information indicating a status detected by a sensor,
wherein the sensor is a conductive path sensor or an environmental sensor, and
wherein the transmitter is configured to transmit the information a distance of more than 0.5 miles.

91. The tracking device of claim 90, wherein the printed antenna, traces, chip landing pads, and battery terminals are printed in a single layer, and integrated into one unified substrate, utilizing a single pass printing method.

92. The tracking device of claim 91, wherein the sensor comprises a conductive path sensor that is printed and integrated into a substrate.

93. The tracking device of claim 90, wherein the energy storage device comprises a printed battery.

94. The tracking device of claim 93, wherein the printed battery is printed on a same substrate as the printed antenna.

95. The tracking device of claim 93, wherein the printed battery comprises a zinc-based battery.

96. The tracking device of claim 90, wherein the transmitter is configured to transmit the information in response to detection of the status by a sensor, in response to expiration of a timer, or both.

97. The tracking device of claim 90, wherein the tracking device is configured to determine its position.

98. The tracking device of claim 90, wherein the tracking device is configured to perform authentication by reading biometric information.

99. The tracking device of claim 90, wherein power is disconnected to a portion of the tracking device until the sensor detects a change in the status, and the tracking device is configured to provide power to the portion of the tracking device based on the detected change in the status.

100. The tracking device of claim 90, wherein the control circuitry is implemented by a programmed processor or dedicated logic circuitry.

101. The tracking device of claim 90, wherein the tracking device is programmable through a wireless interface.

102. The tracking device of claim 90, further comprising a semiconductor die comprising at least a portion of the control circuitry, where the semiconductor die is a bare semiconductor die without its own package.

103. The tracking device of claim 90, wherein the tracking device is configured to detect opening or closing of an integrated conductive connection.

104. The tracking device of claim 90, wherein the sensor is configured to detect opening of an item, removal of contents from an item, or both.

105. The tracking device of claim 90, wherein the sensor comprises a conductive path sensor having a conductive path, and opening or removal of contents from an item is sensed by opening or closing the conductive path.

106. The tracking device of claim 90, wherein the sensor is configured to detect human interaction with a printed item.

107. The tracking device of claim 90, wherein the sensor is an environmental sensor configured to detect an environmental condition.

108. The tracking device of claim 90, wherein a substrate of the tracking device is mechanically flexible.

109. The tracking device of claim 108, wherein the substrate is a thin film substrate.
